(19) United States
(12) Patent Application Publication
(10) Pub. No.: US 2026/0198866 A1
(43) Pub. Date: Jul. 16, 2026

(54) METHODS FOR PREDICTING HOT FLASHES

(71) Applicant: UNIVERSITY OF MASSACHUSETTS, Bostn, MA (US)

(72) Inventors: Michael BUSA, Westborough, MA (US); Nader NAGHAVI, Westborough, MA (US); Thomas COOK, Westborough, MA (US); Robert MARCOTTE, Westborough, MA (US); Ryan TURNER, Westborough, MA (US)

(21) Appl. No.: 19/133,179

(22) PCT Filed: Jan. 5, 2024
(86) PCT No.: PCT/US2024/010451

§ 371 Date: May 27, 2025

Related U.S. Application Data
(60) Provisional application No. 63/478,582, filed on Jan. 5, 2023.

(51) Int. Cl. A61B 5/00; A61B 5/01; A61B 5/0533

(52) U.S. Cl. CPC A61B 5/7275; A61B 5/01; A61B 5/0533; A61B 5/7267; A61B 5/7282

(57) ABSTRACT

An exemplary system and method that can predict or estimate using machine learning based classification the onset of hot flash events or episodes that can be used to trigger the preemptive cooling intervention to prevent the experience of hot flash or to reduce the severity of the symptoms. The machine learning based classification can employ data from electrodermal activity (EDA) sensor measurements to generate a trigger for administration of active cooling via an active wearable device or garment prior to the subjective perception of hot flashes.

### RELATED APPLICATION

This application claims priority to, and the benefit of, U.S. Provisional Patent Application No. 63/478,582, filed Jan. 5, 2023, entitled “Methods for Predicting Hot Flashes,” which is incorporated by reference herein in its entirety.

### GOVERNMENT SUPPORT CLAUSE

This invention was made with government support under Grant No. 1831178 awarded by the National Science Foundation. The government has certain rights in the invention.

### FIELD OF THE INVENTIONS

The present disclosure generally relates to methods and systems for engineering features or parameters from physiological signals for use in diagnostic applications; in particular, the engineering and use of EDA and non-EDA features for use in characterizing hot flash episodes. The features or parameters may also be used for monitoring or tracking, controls of medical devices, or to guide the treatment of hot flashes.

### BACKGROUND

Hot flashes are a form of flushing that is often caused by the changing hormone levels that typically occur with the start of menopause. They are typically experienced as a feeling of intense heat with sweating and rapid heartbeat and can cause significant impairments in quality of life, mood, and subjective sleep quality among midlife women. Hot flashes are reported to be experienced by 70% of menopausal women.

Despite rapid improvements in the field of active wearable devices, including watches, accelerometers, motion sensors, etc., there is a gap in the understanding and usage of wearable devices that can actively work to enhance the thermal comfort of the wearer, including hot flashes.

Current hot flash detection technologies are being investigated and are employed mainly in a research or clinical setting. Certain systems employ an electrodermal activity (EDA) stream collected from transsternal electrodes and evaluate for spikes in the signal, e.g., using a slope threshold (changing >2 uMho over 30 seconds).

It is a benefit to have a system that can mitigate the symptoms of hot flashes and like conditions.

### SUMMARY

An exemplary system and method are provided that can predict and/or detect, using machine learning-based classification, the onset of hot flash events or episodes. This classification can be used to trigger the preemptive cooling intervention to prevent the experience of a hot flash or to reduce the severity of the symptoms. The machine learning-based classification can employ data from electrodermal activity (EDA) sensor measurements to 7 generate a trigger for the administration of active cooling via an active wearable device or garment prior to the subjective onset of hot flashes. Implementations of the machine learning-based classification are observed to have over of >80% identification rate, and <3% false positive rate. The term “predict” is interchangeably used herein with the term “estimate” and refers to the estimating of the offset of a physiological state of interest, e.g., hot flashes, based on observed indications of them. The term “detect” refers to the current observation of the physiological state.

The exemplary system and method can employ EDA rate-of-change evaluation features of the EDA measurement waveform. In some embodiments, the EDA rate-of-change evaluation features employ a median subtracted integral operation that performs an integration operation of a calculated difference between the EDA measurements and median of measurements over a moving window. The EDA rate-of-change features may include curve fit topological analysis of an exponential function (e.g., biexponential function) that is fitted onto a moving window of the EDA waveform.

As used herein, the term “feature” (in the context of machine learning and pattern recognition and as used herein) generally refers to an individual measurable property or characteristic of a phenomenon being observed. A feature is defined by analysis and may be determined in groups in combination with other features from a common model or analytical framework.

In an aspect, a method is disclosed for estimating values of one or more metrics associated with a hot flash episode, the method comprising acquiring, by one or more processors, a data set of a subject comprising one or more physiological signals (e.g., one or more electrodermal activity signals, cardiac signals, etc.); determining, by the one or more processors or a different computing device, one or more values of one or more electrodermal activity features that describe rate-of-change associated properties of the one or more acquired physiological signals; determining, by the one or more processors or the different computing device, an estimated value for the presence of an onset of a hot flash event based on an application of the determined values of the electrodermal activity features to an estimation model (e.g., classifier model, a linear model, a decision tree model, a support vector machine model, a neural network model, etc.); and outputting, by the one or more processors, the estimated value for the prediction or presence of an onset of a hot flash event, wherein the outputted value is presented in a user interface and/or trigger control action of an interventional device to treat or reduce effects of the hot flash event.

In some embodiments, the electrodermal activity features include a median substrate integration analysis that performs an integration operation of difference between measurements and median of measurements over a moving window.

In some embodiments, the electrodermal activity features include a minimum value assessment of a first-order derivative and/or a second-order derivative of an exponential curve fit equation of the one or more physiological signals.

In some embodiments, the electrodermal activity features include a maximum value assessment of a first-order derivative or a second-order derivative of an exponential curve fit equation of the one or more physiological signals.

In some embodiments, the electrodermal activity features include a minimum value assessment, or a maximum value assessment, of a multiplication operation of (i) a first-order derivative of an exponential curve fit equation and (ii) a second-order derivative of an exponential curve fit equation of the one or more physiological signals.

In some embodiments, the estimation model includes at least one of a classifier model, a linear model, a decision tree model, a support vector machine model, a neural network model, and an ensemble model.

In some embodiments, the determining of the estimated value for the presence of an onset of a hot flash event is performed on cloud infrastructure.

In some embodiments, the determining of the estimated value for the presence of an onset of a hot flash event is performed on a wearable device comprising an electrodermal activity sensor.

In some embodiments, the determining of the estimated value for the presence of an onset of a hot flash event is performed on a portable computing device, e.g., smart phone, that connects to a sensor and/or an interventional device.

In some embodiments, the determining of the estimated value for the presence of an onset of a hot flash event is performed on a wearable device comprising the interventional device.

In some embodiments, the physiological signals are acquired via an electrodermal activity (EDA) sensor.

In some embodiments, the physiological signals are acquired via an electrocardiogram, an optical sensor, or a temperature sensor. In some embodiments, the optical sensor includes a photoplethysmographic sensor. In some embodiments, the temperature sensor includes a thermal imaging camera.

In another aspect, a system is disclosed comprising a processor; and a memory having instructions stored thereon, wherein execution of the instructions by the processor causes the processor to perform any one of the above-discussed methods.

In some embodiments, the instructions to estimate values of one or more metrics associated with a hot flash episode are located and implemented on a cloud infrastructure.

In some embodiments, the instructions to estimate values of one or more metrics associated with a hot flash episode are located and implemented on a wearable device or smart garment.

In some embodiments, the instructions to estimate values of one or more metrics associated with a hot flash episode are located and implemented on a local computing device (e.g., smartphone).

In another aspect, a non-transitory computer-readable medium is disclosed, having instructions stored thereon, wherein execution of the instructions by a processor causes the processor to perform any one of the above-discussed methods.

### BRIEF DESCRIPTION OF THE DRAWINGS

The accompanying drawings, which are incorporated in and constitute a part of this specification, illustrate embodiments and, together with the description, serve to explain the principles of the methods and systems.

Embodiments of the present invention may be better understood from the following detailed description when read in conjunction with the accompanying drawings. Such embodiments, which are for illustrative purposes only, depict novel and non-obvious aspects of the invention. The drawings include the following figures:

FIGS. 1A-1H each is a schematic diagram of an example system configured to compute electrodermal activity-related features or parameters that can be used to generate, via a classifier, one or more metrics associated with the physiological state, e.g., relating to hot flashes of a patient or user, to trigger an interventional treatment or action in accordance with an illustrative embodiment.

FIGS. 2A and 2B each show an example method to use physiological signal features or parameters, e.g., EDA-related features or parameters, or their intermediate outputs in a practical application for diagnostics, treatment, monitoring, or tracking of hot flash-related events.

FIGS. 3-4 each shows an example electrodermal activity-related feature computation module configured to determine values of electrodermal activity-rated features or parameters in accordance with an illustrative embodiment.

FIG. 5A shows an example EDA signal.

FIGS. 5B and 5C each shows a sequence of the changes in the feature values of the modules of FIGS. 3 and 4 during a hot flash onset event in accordance with an illustrative embodiment.

FIGS. 6A and 6B show the process employed in a study to develop univariate-feature models and multivariate features models that can predict and/or detect hot flashes.

FIG. 7A shows a flowchart for a univariate model implementation for a closed-loop HF intervention system using a univariate classification model.

FIG. 7B shows an example HF intervention system configured to operate with online model personalization using user feedback.

FIG. 7C shows a flowchart for a closed-loop HF intervention system using a multivariate classification model.

FIG. 7D shows a closed-loop multivariate model implementation with model personalization.

### DETAILED DESCRIPTION

Each and every feature described herein, and each and every combination of two or more of such features, is included within the scope of the present invention, provided that the features included in such a combination are not mutually inconsistent.

While the instant disclosure is directed to the practical assessment of EDA signals, e.g., raw electrical or optical signals, etc., in the diagnosis, tracking, and treatment of the onset of a hot flash event. The assessment may be used in the controls of medical equipment or consumer electronic devices or in monitoring applications.

### Example System #1

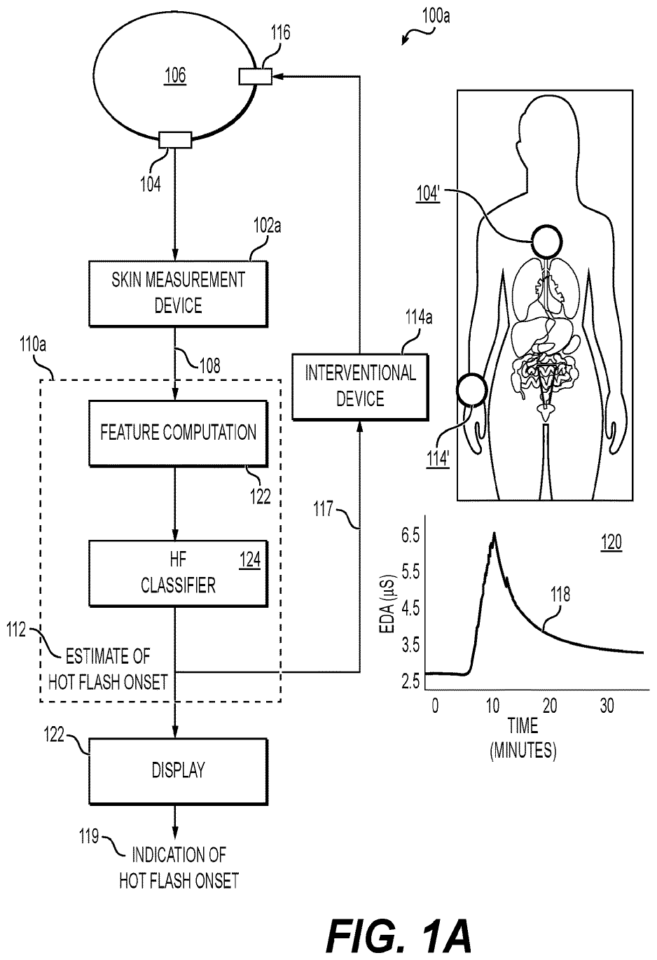

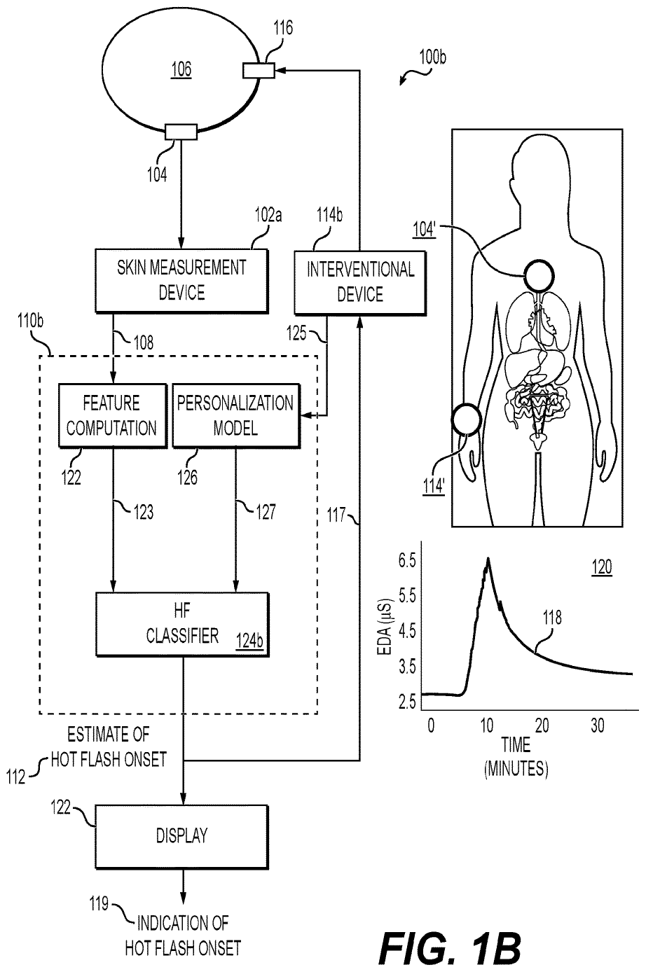

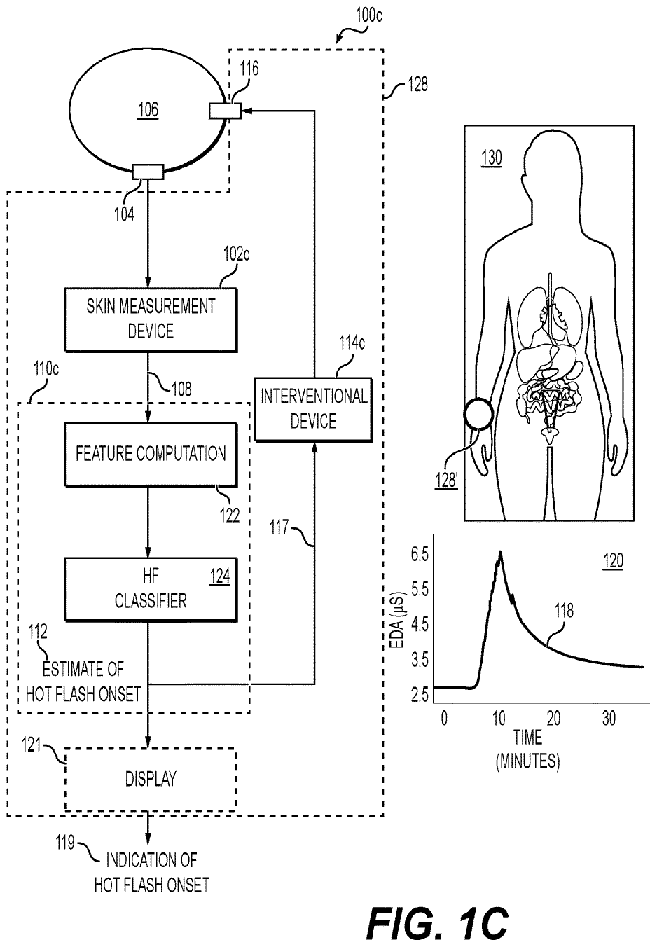

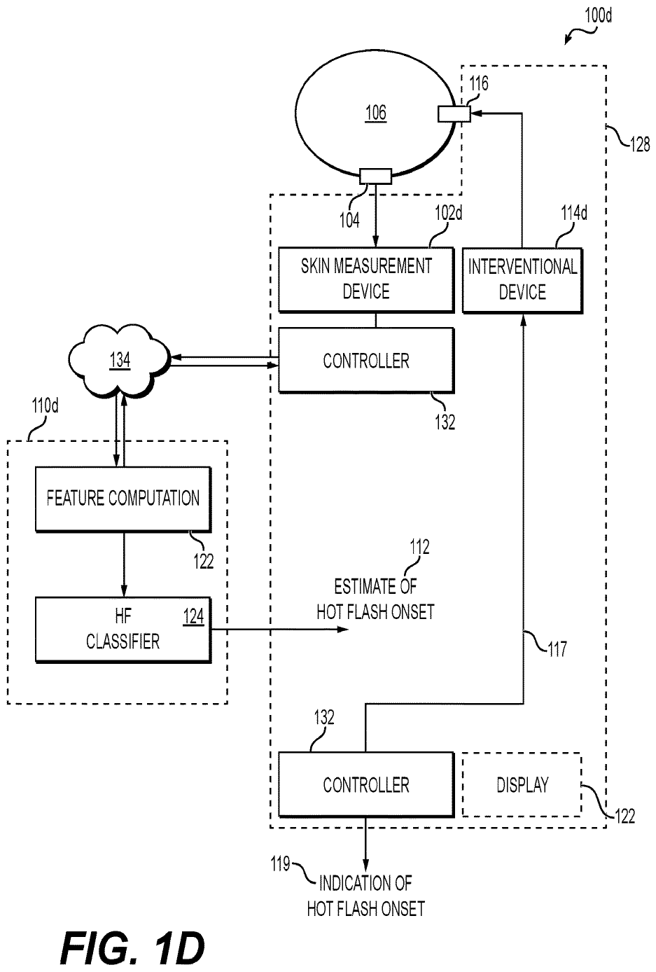

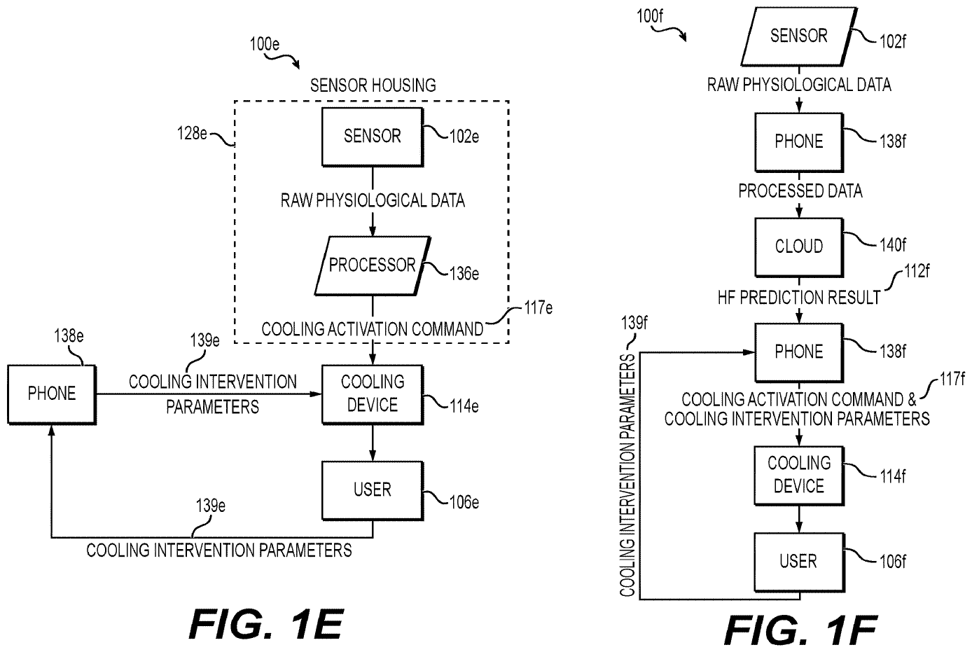

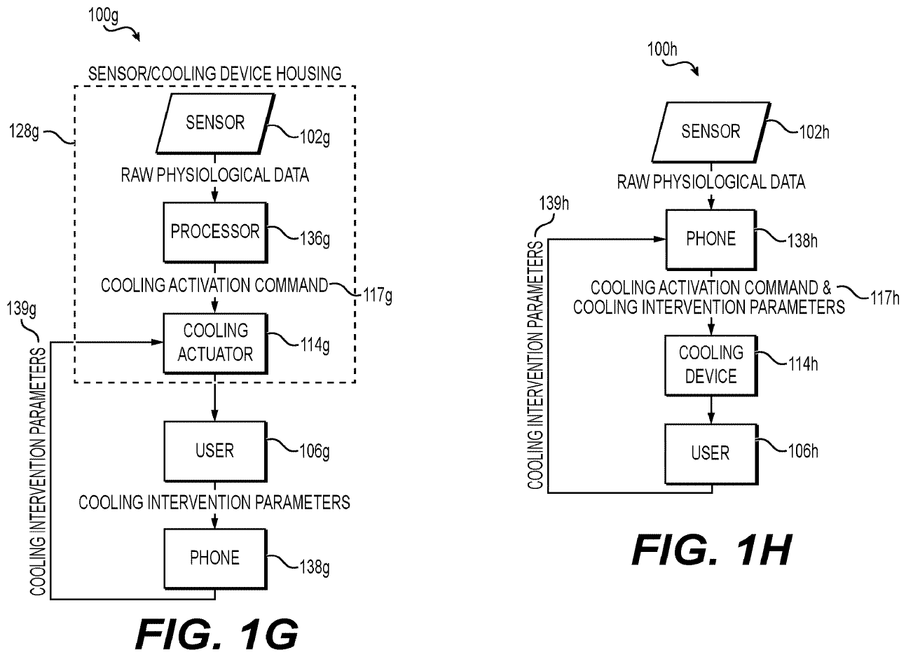

FIG. 1A is a schematic diagram of an example system 100 (shown as 100a) configured to compute electrodermal activity-related features or parameters that can be used to generate, via a set of computing operations, or algorithm, that may or may not include classifier such as a machine-learned classifier, one or more metrics associated with the physiological state, e.g., relating to hot flashes, of a patient or user, to trigger an interventional treatment or action in accordance with an illustrative embodiment. The electrodermal activity-related features or parameters may be used solely or in combination with other features or parameters to generate, via the computing operation or algorithm (e.g., machine-learned classifier), one or more metrics associated with the physiological state. The system 100a may be used in a production application or the development of electrodermal activity-related features and other classes of features. The term “algorithm” refers to a set of pre-defined rules, or logical or mathematical operations, implemented as a set of one or more computing instructions. Algorithms unless stated otherwise can refer to run-time instructions for a given application or simulation instructions for the same.

In the example shown in FIG. 1A, the system 100a includes a skin measurement device 102 (shown as 102a) configured with a skin sensor 104 placed on the skin of a user 106 to provide skin-property associated measurements 108 to an analysis module 110 (shown as 110a) configured to predict and/or detect the onset of hot flashes 112 (shown as “Estimate of hot flash onset” 112) to trigger operations of an interventional device 114 (shown as 114a). In the example shown in FIG. 1A, the interventional device 114a is operatively connected to an active treatment component 116 placed on the skin of a user 106. The terms “user,” “subject,” and “patient” as used herein are generally used interchangeably to refer to those who had undergone analysis performed by the exemplary systems and methods.

The system 100a can be used to monitor the onset of a hot flash episode and trigger the administration of interventional treatment, e.g., active thermal regulation, for the onset of a hot flash episode prior to the subjective onset of hot flashes and without intervention by the user. The interventional treatment could reduce the severity of the symptoms of a hot flash episode. Accurate prediction is beneficial to avoid the false triggers of interventional treatment that would otherwise reduce the operational window of a device, as treatment devices are likely configured with sufficient resources to provide only a pre-defined number of treatments for a given charge, while also minimizing discomfort for the user or patient.

Skin sensor 104 is configured to be placed on the skin of the patient or user 106, e.g., at the wrist, the neck, the back, the torso, and the thigh, among other locations described herein and can provide a measure of electrodermal activity at that location. In the example shown in FIG. 1A, skin sensor 104 (shown as 104′) is shown placed on the sternal region of the torso of the user or patient. Electrodermal activity can include or is also referred to as skin electrical conductance or resistance, potential, impedance, electrochemical skin conductance, admittance, galvanic skin response (GSR), electrodermal response (EDR), psychogalvanic reflex (PGR), skin conductance response (SCR), sympathetic skin response (SSR), and skin conductance level (SCL), e.g., made by electrodes (e.g., to measure conductance, admittance, resistance, impedance, potential, or current), electrochemical sensor, optical, acoustic, thermal, and magnetic, among others described herein. An example electrodermal activity waveform 118 measured at an electrodermal skin sensor (e.g., 104) is shown in diagram 120 during an example hot flash episode. The x-axis shows time in minutes, and the y-axis shows admittance in μS (micro-siemens, μΩ⁻¹).

The onset of hot flashes 112 generally refers to a physiologically observable event that occurs seconds or minutes prior to the event being perceived by the user or patient. The estimation of the onset of hot flashes 112 can provide pre-cooling or early action by the interventional device 114. In some embodiments, the estimation of the onset of hot flashes 112 can be used to initiate action (shown as command 117) once a hot flash episode is observed. In some embodiments, the estimation of the onset of hot flashes 112 can be used to prepare the interventional device 114 to provide the treatment (e.g., pre-cooling). The action provided by the system 100a may be user-definable based on the determined estimation of the onset of hot flashes 112. In some embodiments, the onset of hot flashes 112 is outputted as a notification 119 (shown as “Indication of hot flash onset” 119), e.g., visual notification, via a display 121 operatively coupled to the analysis system 110a. The notification 119 may be used by the user or patient to trigger the interventional device 114a.

Interventional device 114a is configured to provide active regulation, e.g., thermal regulation, of the electrodermal activity of the user or patient. In some embodiments, interventional device 114a is configured to be placed on the skin of the patient or user 106, e.g., at the wrist, the arm, the shoulder, the neck, the back, the torso, the abdomen, the thigh, among other locations described herein and can provide active regulation, e.g., thermal regulation, at that location. In other embodiments, the interventional device 114a may release or inject a hormone or therapeutic on or into the user or patient. In the example shown in FIG. 1A, interventional device 114′ is shown configured as a wearable device, e.g., a watch or a wrist band device with thermal regulation components, that is placed in contact with the wrist region of the user or patient.

Active treatment component 116 may include a cooling plate coupled to a heat-exchanger, Peltier or other thermoelectric devices, active textiles, or endothermic chemical cartridge in which the cooling plate can be placed in contact with the skin of the user or patient to provide cooling to offset the effects of the hot flash episode. In some embodiments, the active treatment component 116 includes networked infrastructure devise, such fans, air conditioners, thermostats, mattresses, among other described herein. In some embodiments, the active treatment component 116 is configured as a dispenser configured to release hormone or therapeutic agents to the skin of the user or patient or through microneedle assemblies.

Analysis module 110a is configured to predict and/or detect the onset of hot flashes 112 based on skin-property associated measurements 108 acquired by the skin sensor 104 of the skin measurement device 102. In the example shown in FIG. 1A, Analysis module 110a includes a feature computation module 122, and a hot flash classifier 124 (shown as “HF Classifier” 124).

Feature computation module 122 includes computer-readable instructions to compute electrodermal activity-related univariate or multivariate feature(s) or parameter(s) that may be used by the hot flash classifier 124, or multiple classifiers. In some embodiments, the electrodermal activity-related feature(s) or parameter(s) includes at least one waveform evaluation analysis and/or topological curve fit analysis of the electrodermal activity measurement waveform (e.g., 118). In some embodiments, the waveform evaluation features or parameters employ a median subtracted integral operation that performs an integration operation of the difference between the measurements and the median of measurements over a moving window. The topological curve fit feature(s) may include one or more outputs of a topological analysis of a function (e.g., exponential-based function, e.g., biexponential function) that is fitted onto a moving window of the EDA waveform.

Feature computation module 122 may include computer-readable instructions to compute other features or parameters that may be used in the hot flash classifier or other classification. The other features or parameters may be determined based on body or skin temperature readings or acquired waveforms, heat flux measurements, heart rate variability measurements, electrocardiographic signals, or waveforms, among others described herein.

The hot flash classifier 124 is a transfer function, look-up table, model, and operator (e.g., decision tree or rules), developed or derived from a machine learning operation. The machine learning operation may include decision trees, random forests, neural networks, linear models, Gaussian processes, nearest neighbor, SVMs, Naïve Bayes, etc.

### Example System #2

FIG. 1B is a schematic diagram of another example system 100 (shown as 100b) configured to compute electrodermal activity-related features or parameters that can be used to generate, via an algorithm or computable instructions (e.g., machine-learned classifier), one or more metrics associated with the physiological state, e.g., relating to hot flashes, of a patient or user, to trigger an interventional treatment or action in accordance with an illustrative embodiment. System 100b includes a user model module 126 (shown as “Personalization Model” 126) configured to receive user input 125 as to the instances that the user or patient initiates the operation of the interventional device 114b. The user model module 126 may compute other features or parameters (e.g., frequency of treatment, time of day, etc.) that may be used in the hot flash classifier or other classification.

In the example shown in FIG. 1B, the hot flash classifier 124 (shown as 124b) is configured to receive the output 123 of the feature computation module 122 and the output 127 of the user model module 126 to generate the predict and/or detect the onset of hot flashes 112.

User-selectable Tuning. The prediction/estimation models can be individualized in some embodiments. In an example, the user model module 126 can allow the end user to tune the models to their preferences. That is, the user can provide inputs that tune the model to be more or less conservative to the application of the automated intervention. Specifically, users can increase/decrease the prediction time and false positive rates of hot flash events to their preferences. These tunings that increase the prediction time may result in corresponding increased false positives, while the decrease in prediction time can decrease false positives. Increasing the sensitivity of the model could lead to an improved prediction rate and timing but may result in a higher false positive rate.

Encoding individual users or properties of generalized characteristics as inputs to the model may adjust systematically the overall model estimates. When individual users are not encoded in the model, the decision for activation based on model output may be made at a population level. The inclusion of individualized features can further optimize the sensitivity/identification/prediction latency trade-off.

Timing and Operation Adjustments. The timing of the activation of the stimulus device can be tuned. The activation of the stimulus can be controlled by the actuation system separate from this disclosure. In one example, the users can select a model with an improved prediction that would predict HF events earlier and activate the intervention up to 60 seconds prior to the subjective perception of the symptom. The stimulus may be activated with the highest intensity as soon as the HF is identified, or the intensity can ramp up gradually so that the users won't feel a rush of coldness on their skin. Alternatively, the users could also set a “delay” parameter that would be the time difference between when the HF event is identified and when the stimulus is activated. This operation could be based on single-point prediction/detection or require a number of consecutive predictions/detections prior to activation.

### Example System #3

FIG. 1C is a schematic diagram of yet another example system 100 (shown as 100c) configured to compute electrodermal activity-related features or parameters that can be used to generate, via a classifier (e.g., machine-learned classifier), one or more metrics associated with the physiological state, e.g., relating to hot flashes, of a patient or user, to trigger an interventional treatment or action in accordance with an illustrative embodiment. The system 100c can be configured as a wearable device configured as an interventional device 114 with integrated sensors.

In the example shown in FIG. 1C, the system 100c includes a skin measurement device 102 (shown as 102c), the analysis module 110 (shown as 110c), and interventional device 114 (shown as 114c) that are located within the housing 128. Diagram 130 shows the wearable device configured as a watch or a wrist band device with thermal regulation components that is placed in contact with the wrist region of the user or patient. As discussed above, the measurement system 102 includes skin sensor 104 that is configured to be placed on the skin of the patient or user 106, e.g., at the wrist, the neck, the back, the torso, and the thigh, among other locations described herein. Housing 128 may be configured appropriate for such sensors.

The system 100c may include the user model module 126, e.g., as described in relation to FIG. 1B.

### Example System #4

FIG. 1D is a schematic diagram of yet another example system 100 (shown as 100d) configured to compute electrodermal activity-related features or parameters that can be used to generate, via a classifier (e.g., machine-learned classifier), one or more metrics associated with the physiological state, e.g., relating to hot flashes, of a patient or user, to trigger an interventional treatment or action in accordance with an illustrative embodiment. The system 100d can be configured as a wearable device configured as an interventional device, or a smart sensor that operates with an interventional device, that operates in conjunction with cloud infrastructure.

In the example shown in FIG. 1D, the system 100d includes a skin measurement device 102 (shown as 102d) and interventional device 114 (shown as 114d) that are located within housing 128. In other embodiments, the interventional device 114d is located in a separate housing. The system 100d includes a controller 132 that includes a network interface to facilitate operation, over a network 134, with cloud infrastructure executing the analysis module 110 (shown as 110d). The analysis module 110d of the cloud infrastructure receives the raw measurement data from controller 132 of the wearable device and computes an prediction and/or detection of the onset of hot flashes 112. The analysis module 110d then transmits the prediction and/or detection to controller 132 of the wearable device, and controller 132 initiates action (shown as command 117) with the interventional device 114d once a hot flash episode is observed. Network interface may include wireless broadband communication or digital or analog cellular service to connect to a cloud infrastructure to provide the analysis operation.

In other embodiments, network interface includes WiFi, Bluetooth, Zigbee, and other near-range network communication interfaces for the network 134 that includes a local network to interface to an edge or local appliance that can provide analysis operation described herein. That is, the network interface may include NFC/wifi communication to an analytical device located in the immediate environment/domicile. The interface can provide connectivity to a cloud infrastructure to provide the analysis operation.

### Example System #5

FIG. 1E is a schematic diagram of yet another example system 100 (shown as 100e) configured to compute electrodermal activity-related features or parameters that can be used to generate, via a classifier (e.g., machine-learned classifier), one or more metrics associated with the physiological state, e.g., relating to hot flashes, of a patient or user, to trigger an interventional treatment or action in accordance with an illustrative embodiment. System 100e can be implemented as an On-Body Network system in which the sensor signal is transmitted to another mobile device over Bluetooth or other local communication, and the signal processing, feature extraction, and classification components are performed on a mobile device. The output control message can be delivered to the actuation system over Bluetooth or the internet. The system 100e can be configured as two sets of standalone wearable or garment devices; one for the interventional device and the other for the sensor device.

In the example shown in FIG. 1E, the system 100e includes a first module in the sensor housing 128 (shown as “Sensor Housing” 128e) comprising a skin measurement device 102 (shown as “Sensor” 102e) configured to provide raw physiological data to a processor 136 (shown as 136e) that executes the analysis module 110, e.g., as described in relation to FIGS. 1A-1D. The processor 136e provides a command 117 (shown as “Cooling activation command” 117e) to an external interventional device 114 (shown as “Cooling Device” 114e) to provide cooling treatment to the user 106 (shown as 106e).

The system 100e includes a second module comprising the interventional device 114e. The controller (not shown) of the interventional device 114e is configured to receive cooling interventional parameters 139 (shown as 139e) from the user 106e via the user computing device 138 (shown as “Phone” 138e) and relay them to the controller of the interventional device 114e. Examples of cooling interventional parameters can include in some examples but are not limited to, cooling settings (e.g., high, medium, low; specific temperature settings; cooling duration, etc.).

### Example System #6

FIG. 1F is a schematic diagram of yet another example system 100 (shown as 100f) configured to compute electrodermal activity-related features or parameters that can be used to generate, via an algorithm (e.g., machine-learned classifier), one or more metrics associated with the physiological state, e.g., relating to hot flashes, of a patient or user, to trigger an interventional treatment or action in accordance with an illustrative embodiment. System 100f can be an Internet-Connected embodiment in which the signal processing, feature extraction, and classification components are deployed in the cloud or internet infrastructure as well as local computing infrastructure described herein. A standalone wearable or garment device configured as an interventional device can operate with the cloud or internet processing infrastructure to treat or alleviate hot flash episodes.

In the example shown in FIG. 1F, the system 100f includes a skin measurement device 102 (shown as “Sensor” 102f) configured to provide raw physiological data to a user computing device 138 (shown as “Phone” 138f). The user computing device 138f transmits the raw physiological data to the cloud infrastructure 140 (shown as “Cloud” 140f) that executes the analysis module 110, e.g., as described in relation to FIGS. 1A-1D. The analysis module of the cloud infrastructure 140f computes an estimation and/or prediction of the onset of hot flashes 112 (shown as “HF prediction result” 112f) and transmits the estimation result 112f to the user computing device 138f. The user computing device 138f provides the command 117 (shown as “Cooling activation command” 117f) to the interventional device 114 (shown as “Cooling Device” 114f) to provide cooling treatment to the user 106 (shown as 106f). The controller (not shown) of the interventional device 114e can receive cooling interventional parameters 139 (shown as 139f) from the user via the user computing device 138 (shown as “Phone” 138f) and relay them to the controller of the interventional device 114f.

### Example System #7

FIG. 1G is a schematic diagram of yet another example system 100 (shown as 100g) configured to compute electrodermal activity-related features or parameters that can be used to generate, via an algorithm (e.g., machine-learned classifier), one or more metrics associated with the physiological state, e.g., relating to hot flashes, of a patient or user, to trigger an interventional treatment or action in accordance with an illustrative embodiment. The system 100g can be configured as a single standalone wearable or garment device configured with both the interventional device and the sensor device. In some embodiments, system 100g can be implemented as a single device with integrated sensing and cooling hardware.

In the example shown in FIG. 1G, the system 100g includes a sensor housing 128 (shown as “Sensor Housing” 128g) comprising a skin measurement device 102 (shown as “Sensor” 102g) configured to provide raw physiological data to a processor 136 (shown as 136g) that executes the analysis module 110, e.g., as described in relation to FIGS. 1A-1D. The processor 136g provides a command 117 (shown as “Cooling activation command” 117g) to the interventional device 114 (shown as “Cooling Actuator” 114g) to provide cooling treatment to the user 106 (shown as 106g). The controller (not shown) of the interventional device 114g is configured to receive cooling interventional parameters 139 (shown as 139g) from the user 106g via the user computing device 138 (shown as “Phone” 138g).

### Example System #8

FIG. 1H is a schematic diagram of yet another example system 100 (shown as 100h) configured to compute electrodermal activity-related features or parameters that can be used to generate, via a classifier (e.g., machine-learned classifier), one or more metrics associated with the physiological state, e.g., relating to hot flashes, of a patient or user, to trigger an interventional treatment or action in accordance with an illustrative embodiment. System 100h can be configured as an “On body” network embodiment in which the data processing is performed on a user's computing device, e.g., a smartphone.

In the example shown in FIG. 1H, the system 100h includes a skin measurement device 102 (shown as “Sensor” 102h) configured to provide raw physiological data to a user computing device 138 (shown as “Phone” 138h). The user computing device 138h executes the analysis module 110, e.g., as described in relation to FIGS. 1A-1D. The analysis module of the user computing device 138h computes an estimation and/or prediction of the onset of hot flashes 112 and provides the command 117 (shown as “Cooling activation command” 117h) to the interventional device 114 (shown as “Cooling Device” 114h) to provide cooling treatment to the user 106 (shown as 106h). The controller (not shown) of the user computing device 138h can receive cooling interventional parameters 139 (shown as 139h) from the user.

### Example Method of Operation

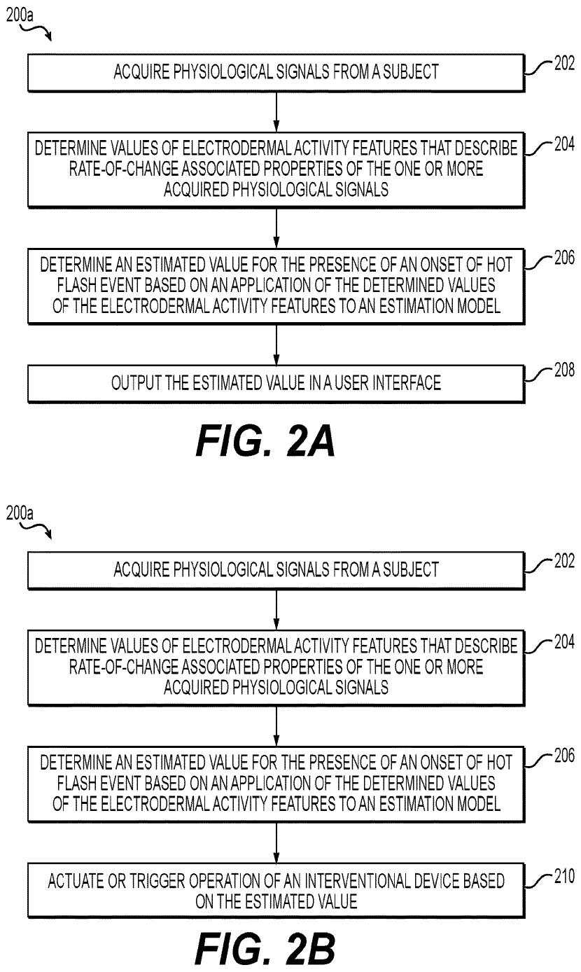

FIGS. 2A and 2B each show an example method 200 (shown as 200a and 200b, respectively) to use physiological signal features or parameters, e.g., EDA-related features or parameters, or their intermediate outputs in a practical application for diagnostics, treatment, monitoring, or tracking of hot flash-related events. In the example shown in FIG. 2A, the detection or prediction of a hot flash-related event is outputted to a display, an audio output, and/or a vibrational output (open loop control) to notify the user of the onset of the hot flash to which the user can take precautionary action or to standby to take such action, e.g., to activate an interventional device. The output may be user selectable.

In the example shown in FIG. 2B, the detection or prediction of a hot flash-related event is outputted to be employed by a controller executing a real-time control loop that triggers an output, or directly actuates the interventional device.

Method 200a includes acquiring (202), by one or more processors, a data set of a subject comprising one or more physiological signals (e.g., one or more electrodermal activity signals, cardiac signals, temperature signals, etc.). In some embodiments, the acquired physiological signals arc transmitted for remote storage and analysis. In other embodiments, the acquired biophysical signals are stored and analyzed locally. The physiological signals may be acquired via wearable devices or smart garments such as EMBR Wave (manufactured by EMBR Labs Inc.) or the smart bra garment (manufactured by Emglare—https://emglare.com/products/bra), among other devices or systems described herein.

Method 200a further includes determining (204), by the one or more processors or a different computing device, one or more values of one or more electrodermal activity (EDA) features or parameters that describe rate-of-change associated properties of the one or more acquired physiological signals. In some embodiments, one or more EDA features or parameters may be used in an analysis. In other embodiments, one or more EDA features or parameters are analyzed in conjunction with non-EDA features in an analysis. Examples of other non-EDA features include skin temperature related features or parameters, core body temperature related features or parameters, heat flux related features or parameters, and/or heart rate and heart-rate variability related features or parameters.

From a ML training/analysis study, it was observed from univariate analyses that using only one EDA feature as described herein when employed in a model can predict and identify 80% and 90% of flashes, respectively, with a very low false positive rate of less than 3%. The results also showed that the best performance achieved with a non-EDA feature was using only one HRV feature when the models could predict and identify 48% and 60% of hot flashes, respectively, although with a high false positive rate of 14%. The best performance achieved using skin temperature features was even lower.

Prediction or Detection Model. Method 200a further includes determining (206), by the one or more processors or the different computing device, an estimated value for the presence of onset of hot flash event based on an application of the determined values of the electrodermal activity features to an estimation model (e.g., classifier model, a linear model, a decision tree model, a support vector machine model, a neural network model, etc.).

Machine Learning. The term “artificial intelligence” (e.g., as used in the context of AI systems) can include any technique that enables one or more computing devices or computing systems (i.e., a machine) to mimic human intelligence. Artificial intelligence (AI) includes but is not limited to knowledge bases, machine learning, representation learning, and deep learning. The term “machine learning” is defined herein to be a subset of AI that enables a machine to acquire knowledge by extracting patterns from raw data. Machine learning techniques include, but are not limited to, logistic regression, support vector machines (SVMs), decision trees, Naïve Bayes classifiers, k-nearest neighbors (kNN), and artificial neural networks. The term “representation learning” is defined herein to be a subset of machine learning that enables a machine to automatically discover representations needed for feature detection, prediction, or classification from raw data. Representation learning techniques include, but are not limited to, autoencoders. The term “deep learning” is defined herein to be a subset of machine learning that enables a machine to automatically discover representations needed for feature detection, prediction, classification, etc., using layers of processing. Deep learning techniques include but are not limited to artificial neural networks or multilayer perceptrons (MLP).

Machine learning models include supervised, semi-supervised, and unsupervised learning models. In a supervised learning model, the model learns a function that maps an input (also known as feature or features) to an output (also known as target or targets) during training with a labeled data set (or dataset). In an unsupervised learning model, the model learns a pattern in the data. In a semi-supervised model, the model learns a function that maps an input (also known as feature or features) to an output (also known as a target) during training with both labeled and unlabeled data.

Neural Networks. An artificial neural network (ANN) is a computing system including a plurality of interconnected neurons (e.g., also referred to as “nodes”). This disclosure contemplates that the nodes can be implemented using a computing device (e.g., a processing unit and memory as described herein). The nodes can be arranged in a plurality of layers, such as an input layer, an output layer, and optionally one or more hidden layers. An ANN having hidden layers can be referred to as a deep neural network or multilayer perceptron (MLP). Each node is connected to one or more other nodes in the ANN. For example, each layer is made of a plurality of nodes, where each node is connected to all nodes in the previous layer. The nodes in a given layer are not interconnected with one another, i.e., the nodes in a given layer function independently of one another. As used herein, nodes in the input layer receive data from outside of the ANN, nodes in the hidden layer(s) modify the data between the input and output layers, and nodes in the output layer provide the results. Each node is configured to receive an input, implement an activation function (e.g., binary step, linear, sigmoid, tan H, or rectified linear unit (ReLU) function), and provide an output in accordance with the activation function. Additionally, each node is associated with a respective weight. ANNs are trained with a dataset to maximize or minimize an objective function. In some implementations, the objective function is a cost function, which is a measure of the ANN's performance (e.g., error such as L1 or L2 loss) during training, and the training algorithm tunes the node weights and/or bias to minimize the cost function. This disclosure contemplates that any algorithm that finds the maximum or minimum of the objective function can be used for training the ANN. Training algorithms for ANNs include but are not limited to backpropagation. It should be understood that an artificial neural network is provided only as an example machine learning model. This disclosure contemplates that the machine learning model can be any supervised learning model, semi-supervised learning model, or unsupervised learning model. Optionally, the machine learning model is a deep learning model. Machine learning models are known in the art and are therefore not described in further detail herein.

A convolutional neural network (CNN) is a type of deep neural network that has been applied, for example, to image analysis applications. Unlike traditional neural networks, each layer in a CNN has a plurality of nodes arranged in three dimensions (width, height, depth). CNNs can include different types of layers, e.g., convolutional, pooling, and fully-connected (also referred to herein as “dense”) layers. A convolutional layer includes a set of filters and performs the bulk of the computations. A pooling layer is optionally inserted between convolutional layers to reduce the computational power and/or control overfitting (e.g., by downsampling). A fully-connected layer includes neurons, where each neuron is connected to all of the neurons in the previous layer. The layers are stacked similar to traditional neural networks.

Other Supervised Learning Models. A logistic regression (LR) classifier is a supervised classification model that uses the logistic function to predict the probability of a target, which can be used for classification. LR classifiers are trained with a data set (also referred to herein as a “dataset”) to maximize or minimize an objective function, for example, a measure of the LR classifier's performance (e.g., an error such as L1 or L2 loss), during training. This disclosure contemplates that any algorithm that finds the minimum of the cost function can be used. LR classifiers are known in the art and are therefore not described in further detail herein.

An Naïve Bayes' (NB) classifier is a supervised classification model that is based on Bayes' Theorem, which assumes independence among features (i.e., the presence of one feature in a class is unrelated to the presence of any other features). NB classifiers are trained with a data set by computing the conditional probability distribution of each feature given a label and applying Bayes' Theorem to compute the conditional probability distribution of a label given an observation. NB classifiers are known in the art and are therefore not described in further detail herein.

A k-NN classifier is a supervised classification model that classifies new data points based on similarity measures (e.g., distance functions). The k-NN classifiers are trained with a data set (also referred to herein as a “dataset”) to maximize or minimize a measure of the k-NN classifier's performance during training. The k-NN classifiers are known in the art and are therefore not described in further detail herein.

A majority voting ensemble is a meta-classifier that combines a plurality of machine learning classifiers for classification via majority voting. In other words, the majority voting ensemble's final prediction (e.g., class label) is the one predicted most frequently by the member classification models. The majority voting ensembles are known in the art and are therefore not described in further detail herein.

Control. Referring to FIG. 2A, Method 200a further includes outputting (208), by one or more processors, the estimated value for the presence of an onset of a hot flash event, wherein the outputted value is presented in a user interface, e.g., of a wearable device such as a smartwatch.

In an alternative embodiment, Method 200b further includes outputting (210), by the one or more processors, the estimated value for the presence of an onset of a hot flash event, wherein the outputted value is employed to trigger control action of an interventional device to treat or reduce effects of the hot flash event. Interventional devices can provide active regulation, e.g., thermal regulation, of the user or the patient. In some embodiments, interventional device 114a is configured to be placed on the skin of the patient or user, e.g., at the wrist, the arm, the shoulder, the neck, the back, the torso, the abdomen, the thigh, among other locations described herein and can provide active regulation, e.g., thermal regulation, of electrodermal activity at that location. In other embodiments, the interventional device may release or inject a hormone or therapeutic on or into the user or patient. An interventional device may be a wearable device, e.g., a watch or a wristband device with thermal regulation components, that is placed in contact with the wrist region of the user or patient. Examples of wearable devices or interventional devices include but are not limited to those described in [6]-[16].

### Electrodermal Activity Feature

In the embodiment of FIGS. 1A-1H, various EDA features or parameters (as embodied in modules 122), as well as non-EDA features, are used by the analysis module 110a to generate one or more metrics associated with the prediction or detection of a hot flash onset of a patient, including EDA curve-fit related features or parameters and EDA variability related features or parameters.

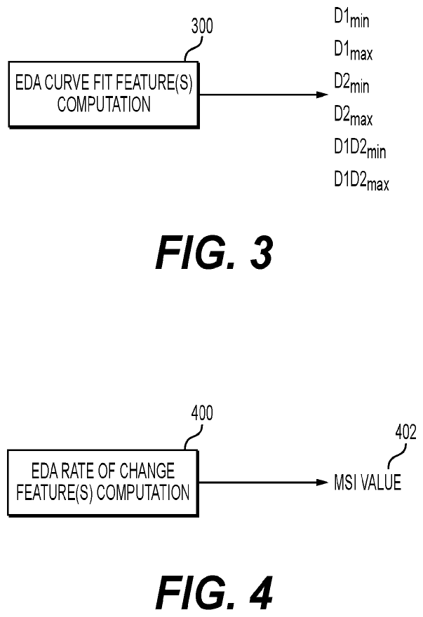

FIGS. 3-4 each shows an example electrodermal activity-related feature computation module, for a total of two example modules, configured to determine values of electrodermal activity-rated features or parameters in accordance with an illustrative embodiment. In particular, the electrodermal activity feature assessment module 300 of FIG. 3 determines features or parameters associated with curve fit from an acquired EDA sensor. Module 400 of FIG. 4 determines features or parameters associated with EDA variability. The analysis module (e.g., 110), more specifically the HF classifier, may call on specific feature functions within any of these modules 300, 400 in whole, or in part as described below for a given application.

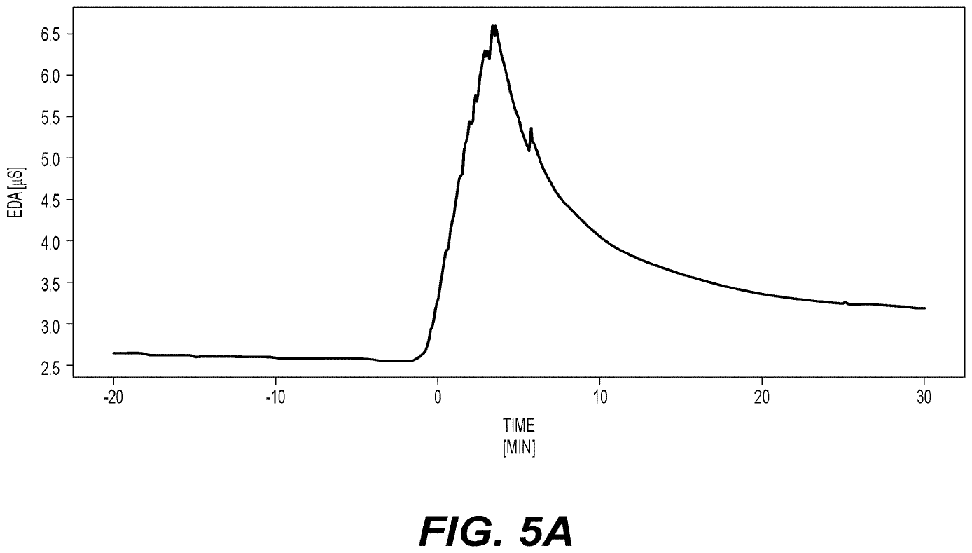

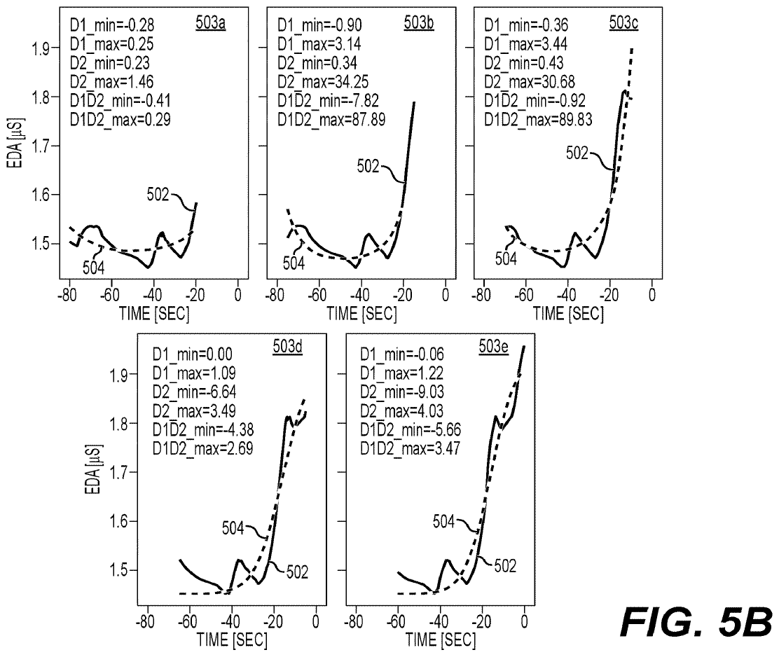

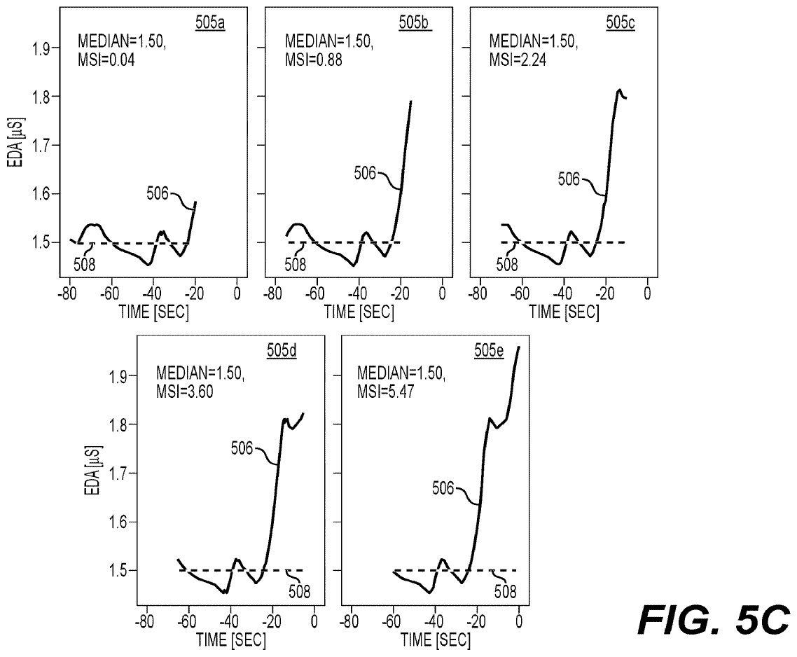

FIG. 5A shows an example EDA signal. EDA-based features can accurately identify a hot flash event based on the change in the shape of an EDA signal, on average, 20 seconds before the subjective perception of the symptom. That is, the system can accurately anticipate or identify hot flash events before a person is aware of the hot flash, and thus the output of the system can be used to deliver “predictive” interventions for hot flash management. The features disclosed can enable the fastest and most accurate detection of hot flash onsets reported in the literature.

Discussion In contrast to traditional hot-flash detection algorithms that employ a threshold analysis, e.g., 2 uS change in EDA amplitude within 30 seconds, the electrodermal activity feature assessment modules 300 and 400 each employ a rate-of-change analysis via a curve fit or an integral of a median subtraction operation. Modules 300, 400 employ a rate of change detector that minimizes or filters false positives within the analysis, e.g., that may be appear as spikes in the acquired signals.

Example #1—EDA Rate-of-Change Estimation via Curve Fit. FIG. 3 illustrates the first of two example feature categories for EDA assessment based on a curve fit to assess the rate of change. In the example shown in FIG. 3, an EDA curve fit feature assessment module 300 is configured to determine output values of EDA-associated curve fit features or parameters that characterize a biexponential equation used to fit an EDA signal of a patient.

The curve-fit class of features employs analysis that fits a biexponential function on the EDA signal to estimate the rate of change. Other exponential or polynomial functions may be employed. For biexponential equations, the equation used to fit the EDA signal may have the form

$${y}_{c} = A {e}^{{Bx}^{2} + Cx}$$

in which x∈[0,1] is a list with the same length as the EDA values. The biexponential function may then fit on a normalized EDA value, yₙ=y−yₘᵢₙ.

Table 1A shows an example set of six extractable rate-of-change characterizations of the EDA signal. The curvefit features denote features created based on an estimated biexponential function fitted to each data window. The features may be standardized using non-parametric methods, using the sample median and estimated interquartile range.

It was observed that each of the features of Table 1A had been experimentally determined via univariate and multivariate analysis to have significant utility in the assessment of the presence or non-presence of the onset of hot flashes.

TABLE 1A

| Feature Name | Feature Description |
|---|---|
| Curvefit_d1_min | Minimum value of the first order derivative of the |
| | biexponential equation, y_{c}′ |
| | d1ₘᵢₙ = min(y_{c}′) |
| Curvefit_d1_max | Maximum value of the first order derivative of the |
| | biexponential equation, y_{c}′ |
| | d1ₘₐₓ = max(y_{c}′) |
| Curvefit_d2_min | Minimum value of the second order derivative of the |
| | biexponential equation |
| | d2ₘᵢₙ = min(y_{c}″) |
| Curvefit_d2_max | Maximum value of the second order derivative of the |
| | biexponential equation, y_{c}″ |
| | d2ₘₐₓ = max(y_{c}″) |
| Curvefit_d1d2_min | Minimum value of the multiplication of the first and second |
| | order derivatives of the biexponential equation |
| | d1d2ₘᵢₙ = min(y_{c}′ × y_{c}″) |
| Curvefit_d1d2_max | Maximum value of the multiplication of the first and second |
| | order derivatives of the biexponential equation |
| | d1d2ₘₐₓ = max(y_{c}′ × y_{c}″) |

FIG. 5B shows a sequence of the changes 503a, 503b, 503c, 503d, and 503e of the curve fit feature values during a hot flash onset event. In each of the subplots of FIG. 5B, the horizontal axis is the time in seconds (t=0 indicates the hot flash onset), and the y-axis is the EDA measurement of admittance value in micro-siemens. The solid lines 502 are the raw EDA measurement, and the dashed lines 504 are the estimated EDA values. In the analysis employed in the study, the features were calculated every 5 seconds (STEP=5) and employed the last 60 seconds (WINDOW=60) of the EDA signal.

Example #2—EDA Rate-of-Change Estimation via Median Substrate Integration. FIG. 4 illustrates the second of two example feature categories for EDA assessment based on the rate of change. In the example shown in FIG. 4, an EDA rate-of-change feature assessment module 400 is configured to determine output values of EDA-associated median-substrate-integrate features or parameters, e.g., per Equation 1, shown as “MSI value” 402.

$$msi = \int ( y - {y}_{m e d} ) dx \qquad ( Eq . 1 )$$

Per Equation 1, module 400 is configured to determine the median value for the current window of physiological signal.

FIG. 5C shows a sequence of the changes 505a, 505b, 505c, 505d, 505e of the MSI rate-of-change feature values preceding a hot flash onset event. In each of the subplots of FIG. 5C, the horizontal axis is the time in seconds (t=0 indicates the hot flash onset), and the y-axis is the EDA measurement of admittance value in micro-siemens. The solid lines 506 are calculated MSI values, and the dashed lines 508 are the median values for the current window. In the study, the optimal window length for the MSI feature extraction is 120 seconds. It can be observed in FIG. 5C that the MSI feature values rise as getting closer to the onset of a HF onset with higher EDA values.

Other Features. Table 1B shows an example set of other extractable EDA and non-EDA features, including for MovisensEDA EDA signals, including the tonic component of EDA signal (EDA-tonic) and the phasic component of EDA signal (EDA-phasic); SKT signals, including all skin temperature channels, including MovisensEDA, MovisensECG, and greenTEG; CBT signals, e.g., greenTEG Core Body Temperature; HFX signals, e.g., greenTEG Heat Flux; HR signals, e.g., Heart Rate; HRV signals, e.g., Hear Rate Variability.

It was observed that each of the features of Table 1B had been experimentally determined via univariate and multivariate analysis to have significant utility in the assessment of the presence or non-presence of the onset of hot flashes. That is, the features have been employed in one or models or algorithms for the assessment of the presence or non-presence of the onset of hot flashes.

TABLE 1B

| Feature Class | Feature Name | Feature Description |
|---|---|---|
| Statistical Features | mean | Mean |
| (extracted for | std | Standard deviation |
| EDA, EDA-tonic, | skew | Skewness |
| EDA-phasic, SKT, | kurtosis | Kurtosis |
| CBT, and HFX) | cv | Coefficient of variation |
| | q25 | First quartile |
| | q50 | Median (second quartile) |
| | q75 | Third quartile |
| | q25_q75 | Interquartile range |
| | msi | Area under the curve of the signal - q50 |
| | min | Minimum |
| | max | Maximum |
| | max_loc | Relative Location (in the range of [0, 1] of |
| | | the maximum value |
| | min_max | Minimum to maximum range |
| Non-linear | sampen | sample entropy |
| Features | | |
| (extracted for | | |
| EDA, EDA-tonic, | | |
| and EDA-phasic) | | |
| Frequency Domain | lf | power in the low frequency band ([0.04, |
| Features | | 0.15] Hz) |
| (extracted only for | hf | power in the high frequency band ([0.15, |
| EDA and HRV) | | 0.40] Hz) |
| | lfhf | ratio lf-to-hf |
| ECG-derived | hr | heart rate |
| Features | sdnn | standard deviation of NN intervals |
| (extracted only for | rmssd | root mean square of successive NN interval |
| HR and HRV) | | differences |
| | sdsd | standard deviation of successive NN |
| | | interval differences |
| | pnn50 | percentage of successive RR intervals that |
| | | differ by more than 50 ms |
| | sd1 | Poincare plot standard deviation |
| | | perpendicular the line of identity |
| | sd2 | Poincare plot standard deviation along the |
| | | line of identity |
| | sd1sd2 | ratio of sd1-to-sd2 |

### Experimental Results and Examples

A study was conducted to develop metrics that can quantitatively measure the capability of machine learning models to predict or detect hot flashes, including the onset of HF events before the subjective perception. In the study, the onset of an HF event is classified as a predicted event if an event was identified within 60 seconds before the subjective perception and as a detected event if the event was identified within 30 seconds after the subjective perception. The number of correctly predicted and detected events divided by the number of total events are defined as prediction and detection rate, respectively. The sum of prediction and detection rates is defined as the “identification rate,” which is a measure of the overall performance of models in the prediction and/or detection of hot flash onsets.

Sensor Data. For the study, data from EDA, ECG, and TEG sensors were employed using an integrated sensor (manufactured by Movisens GmbH). The integrated sensor acquired EDA measurements, heart rate, skin temperature. A second set of integrated sensors (manufactured by GreenTEG) also acquired skin temperature, core body temperature, and heat flux. Data from MovisensEDA, MovisensECG, GreenTEG were acquired from 53 peri- and postmeopausal women, 3×48 h continuous recordings.

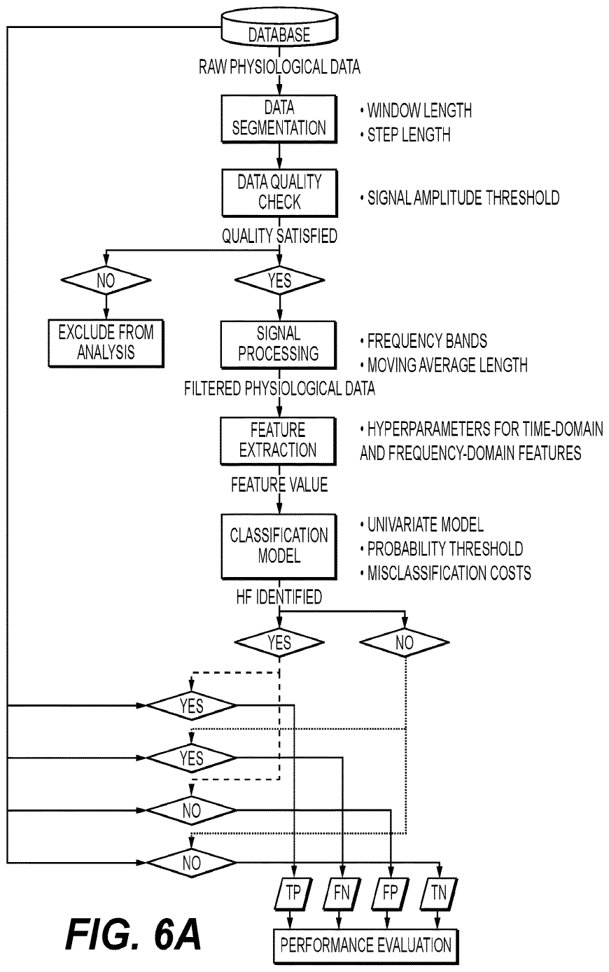

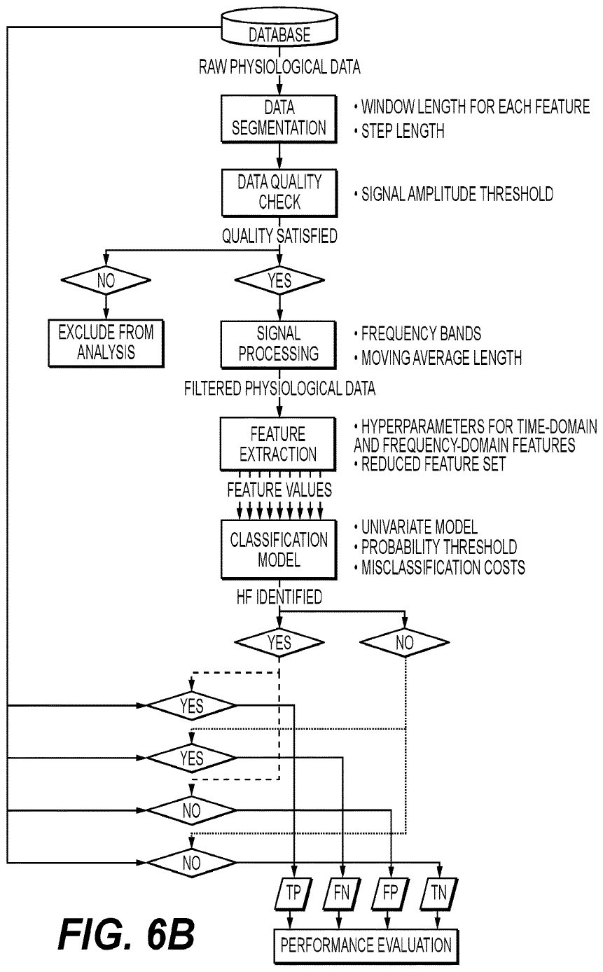

FIGS. 6A and 6B show the process employed to develop univariate-feature models and multivariate features models that can predict or detect hot flashes. The study evaluated multiple rounds of univariate analysis and multivariate analysis to identify the optimal set(s) of features for just-in-time HF identification.

Univariate models (FIG. 6A) are models that use a single feature to identify hot flash onsets. The study tested models from 144 features calculated from all sensors and ranked the models based on prediction rate, identification rate, and false positive rate. These models are of our interest due to their high performance and simplicity of implementation in real-time intervention systems. FIG. 6A shows the flowchart for univariate model training and lists the parameters that were tuned during the optimization process.

Multivariate models are models that use multiple features from either a single or multiple sensors. The advantage of this approach is that it increases the amount of information available to predict hot flashes. The study identified 16 different combinations of features that outperform univariate models in false positive rate as well as prediction and identification rate. FIG. 6B shows the flowchart for training multivariate models and lists the parameters that were tuned during the optimization process.

As shown in FIGS. 6A, 6B, the study preprocessed the data by segmentation operation that defined the training and evaluation data set to a pre-defined length. The study rejected samples that failed a signal amplitude threshold. Similar operations may be implemented in a real-time production system.

The study conducted signal processing analysis to evaluate frequency bands and determine moving average length. For univariate analysis (FIG. 6A), the study employed hyperparameter analysis for time-domain and frequency-domain features. A single feature was employed in univariate model analysis. The study evaluated probability thresholds and misclassification costs. Based on the performance, the study assessed the performance of each model, including true-positive (TP), false-negative (FN), false-positive (FP), and true-negative (TN).

For multivariate analysis (FIG. 6B), the study employed hyperparameter analysis for time-domain and frequency-domain features from a reduced feature set determined to have statistically relevant output. Multiple features from the reduced feature sets were employed in multivariate model analysis. The study evaluated regularization assessment and misclassification costs. Based on the performance, the study assessed the performance of each model, including true-positive (TP), false-negative (FN), false-positive (FP), and true-negative (TN).

Table 2 shows the percentage of the presence of at least one feature from each stream in the final features set for multivariate modeling. In all cases, there has been at least one EDA based feature. The next most common stream was HRV occurring in almost 55% of the final sets. Features from skin temperature streams from chest and/or lateral torso were included in almost 42% of the final sets.

TABLE 2

| Stream | Models Included [ %] |
|---|---|
| Electrodermal Activity | 100 |
| Core Body Temperature | 37.6 |
| Skin Temperature | 41.7 |
| Heat Flux | 32.1 |
| HRV | 54.5 |

The results of the study show that to meet the desired minimum criteria of the study for model performance (>80% identification rate and <3% false positive rate), the classifier model employed at least one EDA feature. In addition, the study results show that the addition of any non-EDA features does not necessarily improve the model performance to the degree that is not achievable using EDA-only features.

The results of the study in the univariate analysis showed that using only one feature from the EDA stream, the models can predict and identify 80% and 90% of flashes, respectively, with a very low false positive rate of less than 3%. The results also showed that the best performance achieved with a non-EDA feature was using only one HRV feature when the models could predict and identify 48% and 60% of hot flashes, respectively, although with a high false positive rate of 14%.

The study additionally identified 6501 models that can satisfy performance criteria of achieving a >70% identification rate, with <10% false positive rate, and prediction times of >5-seconds prior to subjective perception.

Additional examples of models are provided in the Appendix, which is incorporated by reference herein in its entirety.

### EDA Feature Performance

The study evaluated the univariate and multivariate performance of the EDA features of FIGS. 3 and 4. For the characterization, the time-series classification segmented the data into smaller windows in which each window includes sufficient data to inform the occurrence of the symptom within that window. The sliding window approach was used to move the window forward with a defined step. At the end of each step, the features from the corresponding window were extracted and then fed to the classifier. To this end, the classification was executed every step second on the data from the previous window seconds.

Definition. The study compiled the model performance on each of these windows and measured the model performance for sensitivity, specificity, predictivity, prediction rate, identification rate, and identification latency. With the sensitivity and specificity assessment, the study employed binary classification by converting the three-class dataset of non-HF, pre-HF, and post-HF into a binary dataset of non-HF and HF. The pre- and post-HF refer to the respective 60- and 30-second intervals before and after each hot flash onset. With the three-class labels, the study measured the model performance for each class per Table 4 to provide a performance definition as Table 5.

TABLE 4

| Metric | Metric Description |
|---|---|
| Sensitivity | Ratio of correctly classified samples from pre-HF and post-HF classes |
| Specificity | Ratio of correctly classified samples from non-HF class. |
| False | Ratio of incorrectly classified samples from non-HF class (100% - Specificity) |
| Positive Rate | |
| Predictivity | Ratio of correctly classified samples from class pre-HF |
| Prediction | Ratio of correctly predicted HF onsets |
| Rate | |
| Identification | Ratio of correctly identified HF onsets |
| Rate | |
| Identification | Average time difference between the first identified HF sample and the actual |
| Latency | HF onset. A negative value refers to when the first identified HF sample comes |
| | before the actual HF onset (prediction), whereas a positive value refers to when |
| | the first identified HF sample comes after the actual HF onset (detection). |

TABLE 5

| Performance | |
|---|---|
| Metric | Metric Description |
| Correct | A hot flash onset is correctly estimated/predicted, if there is at least one |
| prediction | sample identified as a pre-HF or post-HF class within 60 seconds before the |
| | actual hot flash onset. |
| Correct | A hot flash onset is correctly estimated/detected, if there is at least one |
| detection | sample identified as a pre-HF or post-HF class within 30 seconds after the |
| | actual hot flash onset. |
| Correct | A hot flash onset is correctly estimated/identified, if it was correctly |
| identification | predicted or detected. |

Model assessment. The study investigated the accuracy of HF classification using univariate (single feature) and multivariate (multiple features) models. For univariate model assessment, the study employed logistic regression models. In the logistic regression model, yᵢ∈[0, 1, 2] is assumed as the true label for observation i, and [w₀, w_{0,0}, w₁, w_{0,1}; w₂, w_{0,2}] is the weights matrix where each value wᵢ and w_{0,i} corresponds to class yᵢ. The model aims to predict the class probabilities

$$P ( {y}_{i} = k \vert {x}_{i} ) = \frac{Exp ( {x}_{i} {w}_{k} + {w}_{0 , l} )}{{\sum }_{l = 0}^{2} Exp ( {x}_{i} {w}_{k} + {w}_{0 , l} )}$$

in which the optimal weights matrix W is identified through the optimization process:

$${min}_{W} - C {\sum }_{i = 1}^{n} {\sum }_{k = 0}^{2} [ {y}_{i} = k ] log ( P ( {y}_{i} = k \vert {x}_{i} ) ) + r ( W ) ,$$

where [I] represents the Iverson bracket which evaluates to “0” if [I] is false, otherwise it evaluates to “1.” The regularization term r(W) for L2 penalty is:

$$\frac{1}{2} {‖ W ‖}_{F}^{2} = \frac{1}{2} {\sum }_{i = 0}^{2} {w}_{i}^{2} .$$

For each feature in the dataset, the study fitted a model and measured the model performance. The models trained with curvefit and msi features were observed to have the highest performance among 144 assessed features. Table 6 shows the model performance of the curvefit and msi rate-of-change features under a univariate analysis.

TABLE 6

| | Performance | | | | | |
|---|---|---|---|---|---|---|
| | | | | | | Identifi- |
| | | Sensi- | Predict- | Prediction | Identifi- | cation |
| | Specificity | tivity | ivity | Rate | cation | Latency |
| Feature | [%] | [%] | [%] | [%] | Rate [%] | [sec] |
| msi | 95.9861 | 29.7498 | 22.0889 | 74.5790 | 89.7355 | −20.1 |
| d1ₘᵢₙ | 98.5471 | 9.5420 | 3.2056 | 12.4015 | 49.4472 | 3.2 |
| d1ₘₐₓ | 98.2136 | 33.5839 | 17.2918 | 63.0671 | 78.5706 | −13.4 |
| d2ₘᵢₙ | 98.1931 | 20.3392 | 8.0998 | 33.3286 | 74.8465 | −4.4 |
| d2ₘₐₓ | 98.1405 | 30.8346 | 18.6791 | 69.3162 | 82.7615 | −17.6 |
| d1d2ₘᵢₙ | 98.0783 | 19.0993 | 7.3072 | 25.6375 | 69.4844 | −0.9 |
| d1d2ₘₐₓ | 98.3987 | 31.0440 | 17.1306 | 64.2135 | 79.3673 | −14.4 |

Multivariate Models. For multivariate model assessment, the study employed Linear SVM models. In linear SVM, a training dataset of n points of the form (x₁, y₁), . . . , (xₙ, yₙ) is provided, where yᵢ are either 1 or −1, each indicating the true class to which the point xᵢ belong in which each xᵢ is a p-dimensional vector. The model aims to find the maximum margin hyperplane that divides the group of points xᵢ for which yᵢ=1 from the group of points for which yᵢ=−1 having a maximum distance between the hyperplane and the nearest point xᵢ from either group. The hyperplane can be defined as the set of points x satisfying [symbol] ^{T} [symbol] +b=0, where [symbol] is a vector (e.g., normal vector) to the hyperplane. The classifier can have the form: h(x)=sign( [symbol] ^{T}x+b), with labels {+1, −1}. The classifier parameters w and b can be determined as minimizing the loss function (e.g., hinge loss as

$$[ \frac{1}{n} {\sum }_{i = 1}^{n} max ( 0 , 1 - {y}_{i} ( {𝕨}^{T} 𝕩 + b ) ) ] + {min}_{𝕨 , b} \frac{1}{2} {𝕨}^{T} 𝕨 ) .$$

The study used the recursive feature elimination method to identify the optimal set of features using each set of hyperparameters. The models were trained with a feature set including the curvefit and/or msi rate-of-change features (the study generated 1303 models, with unique feature sets, with Specificity>97%, Prediction Rate>70%, Identification Rate>80%, and Identification Latency of around −21 or −22 seconds demonstrating strong capability for hot flash onset prediction). Table 7 shows model performance for the top 10 models of the 1303 models sorted by Identification Rate. The best performing model (top row with 6 features) is a Linear SVM model including curvefit, low frequency power, interquartile range, and standard deviation features. It can have the form:

$$y = sign ( {w}_{i}^{T} {x}_{i} + b ) ,$$

e.g., w₁*curvefit_d1_max+w₂*curvefit_d1d2_min+w₃*curvefit_d1d2_max+w₄*lf+w₅*q25_q75+w₆*std+b in which the weights wᵢ and bias b are determined through the training.

TABLE 7

| | Number | Performance^{1, 2, 3} | | | |
|---|---|---|---|---|---|
| Model | of | | Specificity | Sensitivity | Predictivity |
| Name | Features | Features⁴ | [%] | [%] | [%] |
| model_01 | 6 | curvefit_d1_max | 97.6382 | 28.0299 | 22.6775 |
| | | curvefit_d1d2_min | | | |
| | | lf | | | |
| | | q25_q75 | | | |
| | | curvefit_d1d2_max⁵ | | | |
| | | std⁵ | | | |
| model_02 | 18 | curvefit_d1_max | 97.3263 | 28.0716 | 22.6079 |
| | | curvefit_d1d2_max | | | |
| | | curvefit_d1d2_min | | | |
| | | curvefit_d2_max | | | |
| | | lf | | | |
| | | max | | | |
| | | min | | | |
| | | min_max | | | |
| | | q25_q75 | | | |
| | | std | | | |
| | | curvefit_d1_max⁵ | | | |
| | | curvefit_d1d2_max⁵ | | | |
| | | curvefit_d1d2_min⁵ | | | |
| | | curvefit_d2_max⁵ | | | |
| | | max⁵ | | | |
| | | min⁵ | | | |
| | | q25⁵ | | | |
| | | std⁵ | | | |
| model_03 | 6 | curvefit_d1d2_min | 97.5339 | 27.8078 | 22.4276 |
| | | lf | | | |
| | | q25_q75 | | | |
| | | curvefit_d1_max⁵ | | | |
| | | curvefit_d1d2_max⁵ | | | |
| | | std⁵ | | | |
| model_04 | 7 | curvefit_d1_max | 97.4093 | 28.2676 | 22.8320 |
| | | curvefit_d1d2_max | | | |
| | | curvefit_d1d2_min | | | |
| | | lf | | | |
| | | q25_q75 | | | |
| | | curvefit_d1d2_max⁵ | | | |
| | | std⁵ | | | |
| model_05 | 8 | curvefit_d1_max | 97.5153 | 28.1474 | 22.7401 |
| | | curvefit_d1d2_max | | | |
| | | curvefit_d1d2_min | | | |
| | | curvefit_d2_max | | | |
| | | lf | | | |
| | | q25_q75 | | | |
| | | curvefit_d1d2_max⁵ | | | |
| | | std⁵ | | | |
| model_06 | 9 | curvefit_d1_max | 97.3958 | 28.2405 | 22.8541 |
| | | curvefit_dld2_max | | | |
| | | curvefit_d1d2_min | | | |
| | | curvefit_d2_max | | | |
| | | lf | | | |
| | | q25_q75 | | | |
| | | curvefit_d1 max⁵ | | | |
| | | curvefit_d1d2_max⁵ | | | |
| | | std⁵ | | | |
| model_07 | 11 | curvefit_d1_max | 97.3654 | 28.1483 | 22.9070 |
| | | curvefit_d1d2_max | | | |
| | | curvefit_d1d2_min | | | |
| | | curvefit_d2_max | | | |
| | | hf | | | |
| | | lf | | | |
| | | min_max | | | |
| | | q25_q75 | | | |
| | | curvefit_d1_max⁵ | | | |
| | | curvefit_d1d2_max⁵ | | | |
| | | std⁵ | | | |
| model_08 | 10 | curvefit_d1_max | 97.4714 | 28.1484 | 22.8983 |
| | | curvefit_d1d2_max | | | |
| | | curvefit_d1d2_min | | | |
| | | curvefit_d2_max | | | |
| | | hf | | | |
| | | lf | | | |
| | | min_max | | | |
| | | q25_q75 | | | |
| | | curvefit_d1d2_max⁵ | | | |
| | | std⁵ | | | |
| model_09 | 9 | curvefit_d1_max | 97.5536 | 27.8890 | 22.5225 |
| | | curvefit_d1d2_min | | | |
| | | lf | | | |
| | | max | | | |
| | | min_max | | | |
| | | q25_q75 | | | |
| | | curvefit_d1d2_max⁵ | | | |
| | | q25⁵ | | | |
| | | std⁵ | | | |
| model_10 | 19 | curvefit_d1_max | 97.3264 | 28.2235 | 22.8120 |
| | | curvefit_d1d2_max | | | |
| | | curvefit_d1d2_min | | | |
| | | curvefit_d2_max | | | |
| | | lf | | | |
| | | max | | | |
| | | min | | | |
| | | min_max | | | |
| | | q25_q75 | | | |
| | | slope_post | | | |
| | | std | | | |
| | | curvefit_d1_max⁵ | | | |
| | | curvefit_d1d2_max⁵ | | | |
| | | curvefit_d1d2_min⁵ | | | |
| | | curvefit_d2_max⁵ | | | |
| | | max⁵ | | | |
| | | min⁵ | | | |
| | | q25⁵ | | | |
| | | std⁵ | | | |

¹Prediction rate is between 71% and 73% for models 01 to 10
²Identification rate is between 84 and 85% for models 01 to 10
³Identification on latency is between −21 and −22 seconds for models 01 to 10.
⁴Input data is based on EDA sensor input unless indicated otherwise.
⁵Input data is based on EDA-tonic data.

Discussion Tables 6 and 7 each shows the models employed in the study outperformed those of results in the literature for detection. According to the inventor's knowledge, the inventors are the first group to successfully attempt a hot flash prediction, and the results outperform the initial expectations of the study. The study identified 1303 models that met the stringent performance criteria of >80% identification rate and <3% false positive rate.

Among these models, the study was able to predict HF events, on average, 21 seconds earlier than the subjective perception of HF onset, and predicted 70% of all HFs. With 6501 models that satisfy the original performance criteria (<10% false positive rate and >70% identification rate, and prediction times of >5 seconds prior to subjective perception), and 1303 models that satisfy much more rigorous criteria (meeting the state-of-the-art false positive rate, of less than 3%, while correctly identifying 80% of the events, and prediction times of >5 seconds prior to subjective perception), the study holds promise in providing a real-time closed-loop interventional implementation. The study employed metrics of performance for real-time usages, such as prediction and identification rates, which are lacking in the literature employing classic model performance measures (e.g., sensitivity, specificity, accuracy, etc.).

The inventors introduced univariate and multivariate models that utilize features from the Electrodermal Activity (EDA) streams that enable hot flash prediction up to 60 seconds prior to the subjective perception of the events. The new features are sensitive to the rapid changes in the signals and are tuned to accurately discriminate pre-HF from non-HF events (i.e., HF prediction). The classifiers trained with these features not only correctly and accurately predict hot flash events but are shown to be outperforming the state-of-the-art by significantly reducing the false positive (non-HF events incorrectly identified as HF) rate.

Indeed, prediction and/or detection can be made from EDA sensor data prior to their subjective onset. The prediction and/or detection can be made, in some embodiments, using EDA sensor data acquired at the lateral torso, or other sensor described herein, which could be more discrete and less bothersome as compared to sternal EDA measurements, though either one can be used or their combination.

There is a commercial interest in detecting and predicting hot flash events using wearable technology. Several companies are evaluating hot flash prediction solutions based on wrist-worn devices. These manufacturers employ different combinations of physiological data streams.

In contrast, the models developed in the study can operate using only a single data stream (e.g., EDA) and a single feature (e.g., the EDA feature described herein) to predict hot flashes. By adding more data streams to the models, the accuracy can be improved, i.e., more predicted hot flash events with reduced false positive rates, at a trade-off of additional sensors.

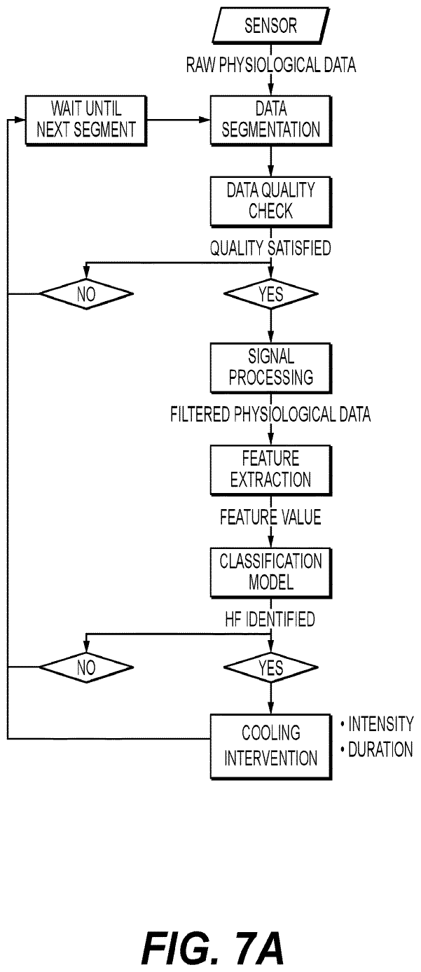

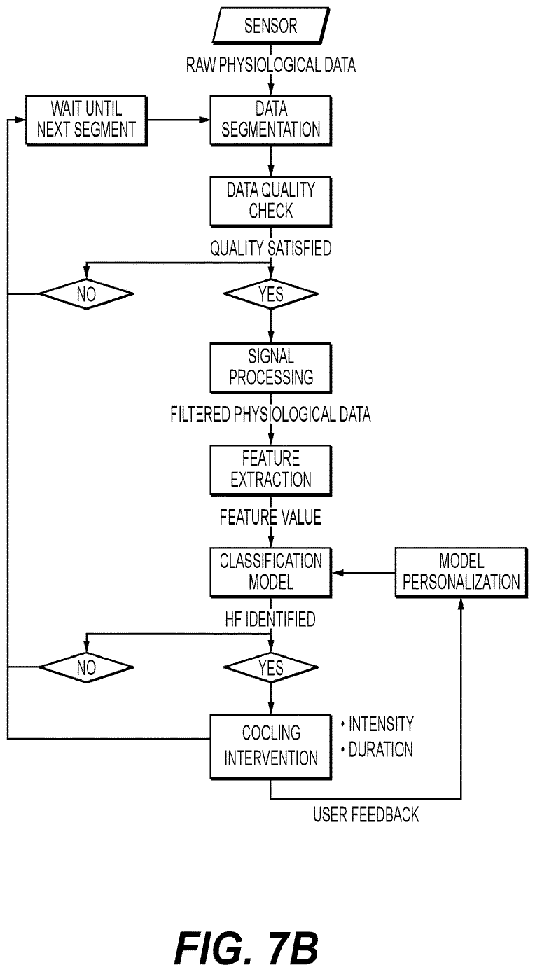

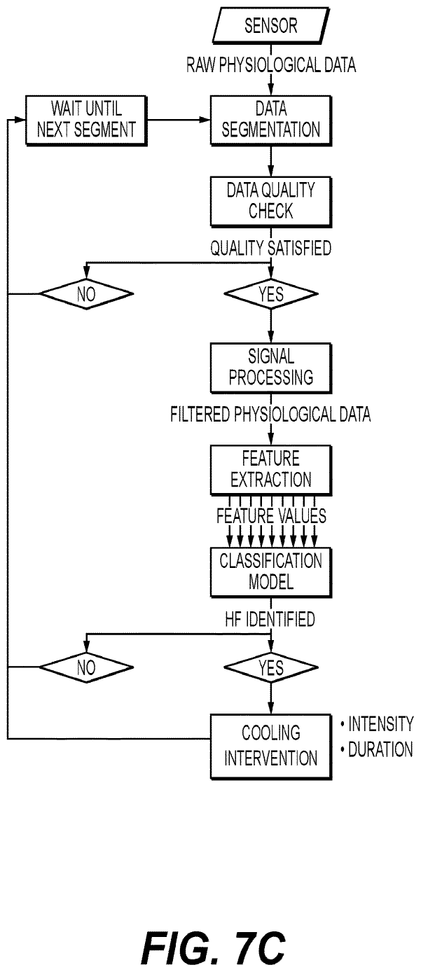

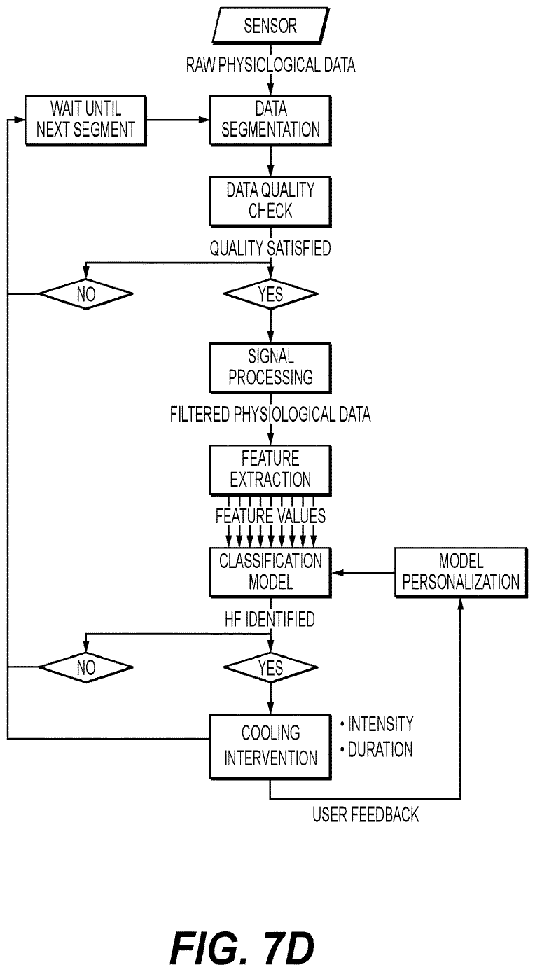

Implementation of Automated Vasomotor Symptom Attenuation. The study also conducted the development and evaluation of an automated vasomotor symptom system or method. FIG. 7A shows a flowchart for a univariate model implementation for a closed-loop HF intervention system using a univariate classification model. FIG. 7B shows an example HF intervention system with online model personalization using user feedback.

The study also developed a multivariate model implementation for automated therapies. FIG. 7C shows a flowchart for a closed-loop HF intervention system using a multivariate classification model. In FIG. 7C, the system includes an HF intervention system configured to operate with an online model personalization from a multi-sensor system using user feedback. FIG. 7D shows a closed-loop multivariate model implementation with model personalization.

Although example embodiments of the present disclosure are explained in some instances in detail herein, it is to be understood that other embodiments are contemplated. Accordingly, it is not intended that the present disclosure be limited in its scope to the details of construction and arrangement of components set forth in the following description or illustrated in the drawings. The present disclosure is capable of other embodiments and of being practiced or carried out in various ways.

The exemplary system and method may be combined with other forms of external stimulus. It could provide delivery notifications to a smartwatch or smart phone, prompt a guided breathing exercise, adjust temperature setpoint of a temperature-controlled system interacting with the user, e.g., a house thermostat, a temperature-controlled mattress, a heated and cooled office chair. ‘The exemplary system and method can be employed to increase convection around the user (e.g., activating a fan, opening a window). In some embodiments, the exemplary system and method can be employed to activate a wearable cooling technology.

The detecting device/system may be implemented as a distinct system from the actuating device/system so long as there is communication between the two systems. This specification may be determined by the technical requirements of the specific data stream used for prediction/detection as well as the type of intervention being delivered. For example, a device that delivers an on-body cooling intervention may have a different capacity for integration with sensing technology than one that provides information to the patient or controls an environmental intervention device (e.g., a fan or HVAC system). In an example implementation, the detecting device may be designed to be wearable and integrated with a wearable actuation device, such that a detecting and actuating system can be delivered in a single device.

It must also be noted that, as used in the specification and the appended claims, the singular forms “a,” “an,” and “the” include plural referents unless the context clearly dictates otherwise. Ranges may be expressed herein as from “about” or “approximately” one particular value and/or to “about” or “approximately” another particular value. When such a range is expressed, other exemplary embodiments include the one particular value and/or to the other particular value.

By “comprising” or “containing” or “including” is meant that at least the name compound, element, particle, or method step is present in the composition or article or method but does not exclude the presence of other compounds, materials, particles, method steps, even if the other such compounds, material, particles, method steps have the same function as what is named.

Example Computing System. The exemplary system and method may be implemented (1) as a sequence of computer-implemented acts or program modules running on a computing system and/or (2) as interconnected machine logic circuits or circuit modules within the computing system. The implementation is a matter of choice dependent on the performance and other requirements of the computing system. Accordingly, the logical operations described herein are referred to variously as state operations, acts, or modules. These operations, acts, and/or modules can be implemented in software, in firmware, in special purpose digital logic, in hardware, and any combination thereof. It should also be appreciated that more or fewer operations can be performed than shown in the figures and described herein. These operations can also be performed in a different order than those described herein.

The computer system is capable of executing the software components described herein for the exemplary method or systems. In an embodiment, the computing device may comprise two or more computers in communication with each other that collaborate to perform a task. For example, but not by way of limitation, an application may be partitioned in such a way as to permit concurrent and/or parallel processing of the instructions of the application. Alternatively, the data processed by the application may be partitioned in such a way as to permit concurrent and/or parallel processing of different portions of a data set by the two or more computers. In an embodiment, virtualization software may be employed by the computing device to provide the functionality of a number of servers that are not directly bound to the number of computers in the computing device. For example, virtualization software may provide twenty virtual servers on four physical computers. In an embodiment, the functionality disclosed above may be provided by executing the application and/or applications in a cloud computing environment. Cloud computing may comprise providing computing services via a network connection using dynamically scalable computing resources. Cloud computing may be supported, at least in part, by virtualization software. A cloud computing environment may be established by an enterprise and/or can be hired on an as-needed basis from a third-party provider. Some cloud computing environments may comprise cloud computing resources owned and operated by the enterprise as well as cloud computing resources hired and/or leased from a third-party provider.

The processing unit may be a standard programmable processor that performs arithmetic and logic operations necessary for the operation of the computing device. While only one processing unit is shown, multiple processors may be present. As used herein, processing unit and processor refers to a physical hardware device that executes encoded instructions for performing functions on inputs and creating outputs, including, for example, but not limited to, microprocessors (MCUs), microcontrollers, graphical processing units (GPUs), and application-specific circuits (ASICs). Thus, while instructions may be discussed as executed by a processor, the instructions may be executed simultaneously, serially, or otherwise executed by one or multiple processors. The computing device may also include a bus or other communication mechanism for communicating information among various components of the computing device.

Computing devices may have additional features/functionality. For example, the computing device may include additional storage, such as removable storage and non-removable storage, including, but not limited to, magnetic or optical disks or tapes. Computing devices may also contain network connection(s) that allow the device to communicate with other devices, such as over the communication pathways described herein. The network connection(s) may take the form of modems, modem banks, Ethernet cards, universal serial bus (USB) interface cards, serial interfaces, token ring cards, fiber distributed data interface (FDDI) cards, wireless local area network (WLAN) cards, radio transceiver cards such as code division multiple access (CDMA), global system for mobile communications (GSM), long-term evolution (LTE), worldwide interoperability for microwave access (WiMAX), and/or other air interface protocol radio transceiver cards, and other well-known network devices. Computing devices may also have input device(s) such as keyboards, keypads, switches, dials, mice, trackballs, touch screens, voice recognizers, card readers, paper tape readers, or other well-known input devices. Output device(s) such as printers, video monitors, liquid crystal displays (LCDs), touch screen displays, displays, speakers, etc., may also be included. The additional devices may be connected to the bus in order to facilitate the communication of data among the components of the computing device. All these devices are well known in the art and need not be discussed at length here.

The processing unit may be configured to execute program code encoded in tangible, computer-readable media. Tangible, computer-readable media refers to any media that is capable of providing data that causes the computing device (i.e., a machine) to operate in a particular fashion. Various computer-readable media may be utilized to provide instructions to the processing unit for execution. Example tangible, computer-readable media may include but is not limited to volatile media, non-volatile media, removable media, and non-removable media implemented in any method or technology for storage of information such as computer-readable instructions, data structures, program modules, or other data. System memory, removable storage, and non-removable storage are all examples of tangible computer storage media. Example of tangible, computer-readable recording media include, but are not limited to, an integrated circuit (e.g., field-programmable gate array or application-specific IC), a hard disk, an optical disk, a magneto-optical disk, a floppy disk, a magnetic tape, a holographic storage medium, a solid-state device, RAM, ROM, electrically erasable program read-only memory (EEPROM), flash memory or other memory technology, CD-ROM, digital versatile disks (DVD) or other optical storage, magnetic cassettes, magnetic tape, magnetic disk storage or other magnetic storage devices.

The following patents, applications, and publications as listed below and throughout this document are hereby incorporated by reference in their entirety herein.

### LIST OF REFERENCES

[1] Forouzanfar M, Zambotti M, Goldstone A, Baker F C. Automatic Detection of Hot Flash Occurrence and Timing from Skin Conductance Activity. Annu Int Conf IEEE Eng Med Biol Soc. 2018 July; 2018:1090-1093. doi: 10.1109/EMBC.2018.8512492. PMID: 30440580; PMCID: PMC7477890.
[2] Thurston R C, Matthews K A, Hernandez J, De La Torre F. Improving the performance of physiologic hot flash measures with support vector machines. Psychophysiology. 2009 March; 46(2): 285-92. doi: 10.1111/j.1469-8986.2008.00770.x. Epub 2009 Jan. 26. PMID: 19170952; PMCID: PMC2755219.
[3] Luo J, Mao A, Zeng Z. Sensor-Based Smart Clothing for Women's Menopause Transition Monitoring. Sensors (Basel). 2020 Feb. 17; 20(4): 1093. doi: 10.3390/s20041093. PMID: 32079349; PMCID: PMC7071038.
[4] Bahr D E, Webster J G, Grady D, Kronenberg F, Creasman J, Macer J, Shults M, Tyler M, Zhou X. Miniature ambulatory skin conductance monitor and algorithm for investigating hot flash events. Physiol Meas. 2014 February; 35(2): 95-110. doi: 10.1088/0967-3334/35/2/95. Epub 2014 Jan. 7. PMID: 24398586; PMCID: PMC3951394.
[5] Bahr D E, Webster J G, Grady D, Kronenberg F, Creasman J, Macer J, Shults M, Tyler M, Zhou X. Miniature ambulatory skin conductance monitor and algorithm for investigating hot flash events. Physiol Meas. 2014 February; 35(2): 95-110. doi: 10.1088/0967-3334/35/2/95. Epub 2014 Jan. 7. PMID: 24398586; PMCID: PMC3951394.
[6] WO2021115890A1
[7] US20220000199A1
[8] US20200013511A1
[9] US2022096317 (EMBR LABS INC)
[10] US2015101788 (EMBR LABS INC)
[11] US2020050248 (EMBR LABS INC)
[12] US2021399186 (EMBR LABS INC)
[13] WO21257394 (EMBR LABS INC)
[14] US2020352777 (EMBR LABS INC)
[15] US2019110950 (EMBR LABS INC)
[16] US2018042761 (EMBR LABS INC)

1. A method for estimating values of one or more metrics associated with a hot flash episode, the method comprising:
acquiring, by one or more processors, a data set of a subject comprising one or more physiological signals;
determining, by the one or more processors or a different computing device, one or more values of one or more electrodermal activity features that describe rate-of-change associated properties of the one or more acquired physiological signals;
determining, by the one or more processors or the different computing device, an estimated value for the presence of an onset of a hot flash event based on an application of the determined values of the electrodermal activity features to an estimation model; and
outputting, by the one or more processors, the estimated value for the presence of an onset of a hot flash event, wherein the outputted value is presented in a user interface and/or trigger control action of an interventional device to treat or reduce effects of the hot flash event.

2. The method of claim 1, wherein the electrodermal activity features include a median substrate integration analysis that performs an integration operation of difference between an instant measurement and a calculated moving-window median.

3. The method of claim 1, wherein the electrodermal activity features include a minimum value assessment of a first-order derivative or a second-order derivative of an exponential curve fit equation of the one or more physiological signals.

4. The method of claim 1, wherein the electrodermal activity features include a maximum value assessment of a first-order derivative or a second-order derivative of an exponential curve fit equation of the one or more physiological signals.

5. The method of claim 1, wherein the electrodermal activity features include a minimum value assessment, or a maximum value assessment, of a multiplication operation of (i) a first-order derivative of an exponential curve fit equation and (ii) a second-order derivative of an exponential curve fit equation of the one or more physiological signals.

6. The method of claim 1, wherein the estimation model includes at least one of a classifier model, a linear model, a decision tree model, a support vector machine model, and a neural network model.

7. The method of claim 1, wherein the determining of the estimated value for the presence of an onset of a hot flash event is performed on cloud infrastructure.

8. The method of claim 1, wherein the determining of the estimated value for the presence of an onset of a hot flash event is performed on a wearable device comprising an electrodermal activity sensor.

9. The method of claim 1, wherein the determining of the estimated value for the presence of an onset of a hot flash event is performed on a wearable device comprising the interventional device.

10. The method of claim 1, wherein the physiological signals are acquired via an electrodermal activity (EDA) sensor.

11. The method of claim 1, wherein the physiological signals are acquired via an electrocardiogram, an optical sensor, or a temperature sensor.

12. A system comprising:
one or more processors; and
a memory having instructions to estimate values of one or more metrics associated with a hot flash episode stored thereon, wherein execution of the instructions by the one or more processors causes the one or more processors to:
acquire, by the one or more processors, a data set of a subject comprising one or more physiological signals;
determine, by the one or more processors or a different computing device, one or more values of one or more electrodermal activity features that describe rate-of-change associated properties of the one or more acquired physiological signals;
determine, by the one or more processors or the different computing device, an estimated value for the presence of an onset of a hot flash event based on an application of the determined values of the electrodermal activity features to an estimation model; and
output, by the one or more processors, the estimated value for the presence of an onset of a hot flash event, wherein the outputted value is presented in a user interface and/or trigger control action of an interventional device to treat or reduce effects of the hot flash event.

13. The system of claim 12, wherein the instructions to estimate values of one or more metrics associated with a hot flash episode are located and implemented on a cloud infrastructure.

14. The system of claim 12, wherein the instructions to estimate values of one or more metrics associated with a hot flash episode are located and implemented on a wearable device or smart garment.

15. The system of claim 12, wherein the instructions to estimate values of one or more metrics associated with a hot flash episode are located and implemented on a local computing device.

16. A non-transitory computer-readable medium having instructions to estimate values of one or more metrics associated with a hot flash episode stored thereon, wherein execution of the instructions by one or more processors causes the one or more processors to:
acquire, by the one or more processors, a data set of a subject comprising one or more physiological signals;
determine, by the one or more processors or a different computing device, one or more values of one or more electrodermal activity features that describe rate-of-change associated properties of the one or more acquired physiological signals;
determine, by the one or more processors or the different computing device, an estimated value for the presence of an onset of a hot flash event based on an application of the determined values of the electrodermal activity features to an estimation model; and
output, by the one or more processors, the estimated value for the presence of an onset of a hot flash event, wherein the outputted value is presented in a user interface and/or trigger control action of an interventional device to treat or reduce effects of the hot flash event.

17. The non-transitory computer-readable medium of claim 16, wherein the electrodermal activity features include a median substrate integration analysis that performs an integration operation of difference between an instant measurement and a calculated moving-window median.

18. The non-transitory computer-readable medium of claim 16, wherein the electrodermal activity features include a minimum value assessment of a first-order derivative or a second-order derivative of an exponential curve fit equation of the one or more physiological signals.

19. The non-transitory computer-readable medium of claim 16, wherein the electrodermal activity features include a maximum value assessment of a first-order derivative or a second-order derivative of an exponential curve fit equation of the one or more physiological signals.

20. The non-transitory computer-readable medium of claim 16, wherein the estimation model includes at least one of a classifier model, a linear model, a decision tree model, a support vector machine model, and a neural network model.
